SEQUENCE LISTING (8 sequences)

SEQ ID NO: 1 - DNA, 20 bp, synthetic construct
  source 1..20: mol_type = other DNA
ccgagtacctgaaaggcgaa

SEQ ID NO: 2 - DNA, 20 bp, synthetic construct
  source 1..20: mol_type = other DNA
ctaatcagggctaagggcga

SEQ ID NO: 3 - DNA, 21 bp, synthetic construct
  source 1..21: mol_type = other DNA
gtgtggaaccgcatcattgag

SEQ ID NO: 4 - DNA, 21 bp, synthetic construct
  source 1..21: mol_type = other DNA
gtctttgagcaccagaggaca

SEQ ID NO: 5 - DNA, 20 bp, synthetic construct
  source 1..20: mol_type = other DNA
gcaccttcacaggcgagacc

SEQ ID NO: 6 - DNA, 20 bp, synthetic construct
  source 1..20: mol_type = other DNA
gaggccctggatacgcgaac

SEQ ID NO: 7 - DNA, 21 bp, synthetic construct
  source 1..21: mol_type = other DNA
tccatgacaactttggcattg

SEQ ID NO: 8 - DNA, 21 bp, synthetic construct
  source 1..21: mol_type = other DNA
cagtcttctgggtggcagtga

(19) United States
(12) Patent Application Publication
(10) Pub. No.: US 2026/0345532 A1
(43) Pub. Date: Nov. 5, 2026

(54) COMPOSITIONS AND METHODS FOR TREATING TACTILE ALLODYNIA IN KABUKI SYNDROME

(71) Applicant: UNIVERSITY OF NOTRE DAME DU LAC, South Bend, IN (US)

(72) Inventors: Kasturi HALDAR, South Bend, IN (US); Md. Suhail ALAM, South Bend, IN (US); Arpitha MYSORE RAJESHEKARA, South Bend, IN (US)

(21) Appl. No.: 19/660,928

(22) Filed: Apr. 28, 2026

Related U.S. Application Data
(60) Provisional application No. 63/796,881, filed on Apr. 29, 2025.

(51) Int. Cl. A61K 31/167; A61K 47/10; A61K 47/20; A61K 47/40; A61P 25/28

(52) U.S. Cl. CPC A61K 31/167; A61K 47/10; A61K 47/20; A61K 47/40; A61P 25/28

(57) ABSTRACT

Described herein is a method of treating tactile allodynia in a subject having Kabuki Syndrome. Also described herein is a method of improving one or more of cognition, memory, sensory responses, or learning in a subject having Kabuki Syndrome. The methods comprise administering to a subject a therapeutically effective amount of a composition comprising a histone deacetylase inhibitor, a polyethylene glycol (PEG), a cyclodextrin, and dimethyl sulfoxide (DMSO). In certain embodiments, the composition may comprise vorinostat, PEG-400, 2-hydroxypropyl-β-cyclodextrin (HP-β-CD), and DMSO.

### CROSS-REFERENCE TO RELATED APPLICATION(S)

This application claims priority to U.S. Provisional Patent Application No. 63/796,881, filed on Apr. 29, 2025, which is incorporated by reference herein in its entirety.

### REFERENCE TO SEQUENCE LISTING

This application was filed with a Sequence Listing XML in ST.26 XML format in accordance with 37 C.F.R. § 1.831 and PCT Rule 13ter. The Sequence Listing XML file submitted in the USPTO Patent Center, “092012-0078-US02_sequence_listing_XML_24 Apr. 2026.xml,” was created on Apr. 24, 2026, contains 8 sequences, has a file size of 8.01 kilobytes (8,208 bytes), and is incorporated by reference in its entirety into the specification.

### BACKGROUND

Kabuki Syndrome (KS) is a multi-system disorder that compromises regulatory mechanisms underlying hypoxia. Varying degrees of intellectual disability appear to be associated with the majority of patients. KS is also associated with defects in cardiac and immunological conditions as well as hippocampal function. Since Kabuki mice show defect in the hippocampal function of tactile allodynia (in other words sensory response to mechanical stimuli), KS provides a monogenetic disorder to query this post-natal function in the brain.

What are needed are compositions, methods, and therapies for treating tactile allodynia and improving cognition, memory, sensory responses, and learning in subjects having Kabuki Syndrome.

### SUMMARY

One embodiment described herein is a method of treating tactile allodynia in a subject having Kabuki Syndrome, the method comprising administering to the subject a therapeutically effective amount of a composition comprising a histone deacetylase inhibitor, a polyethylene glycol (PEG), a cyclodextrin, and dimethyl sulfoxide (DMSO), thereby treating the tactile allodynia in the subject. In one aspect, the histone deacetylase inhibitor comprises vorinostat, belinostat, romidepsin, dacinostat, panobinostat, givinostat, pyroxamide, trichostatin A, m-carboxycinnamic acid bis-hydroxamide (CBHA), or combinations thereof. In another aspect, the histone deacetylase inhibitor comprises vorinostat. In another aspect, the PEG has an average molecular weight of about 200 Da to about 600 Da. In another aspect, the PEG comprises PEG-400. In another aspect, the cyclodextrin has an average molecular weight of about 900 Da to about 1500 Da. In another aspect, the cyclodextrin comprises 2-hydroxypropyl-β-cyclodextrin (HP-β-CD), dimethyl-β-cyclodextrin, hydroxypropyl-α-cyclodextrin, hydroxypropyl-γ-cyclodextrin, or combinations thereof. In another aspect, the cyclodextrin comprises 2-hydroxypropyl-β-cyclodextrin (HP-β-CD). In another aspect, the composition comprises vorinostat, PEG-400, 2-hydroxypropyl-β-cyclodextrin (HP-β-CD), and DMSO. In another aspect, the composition comprises about 10 wt % to about 40 wt % histone deacetylase inhibitor; about 15 wt % to about 50 wt % PEG; about 20 wt % to about 50 wt % cyclodextrin; and about 5 wt % to about 10 wt % DMSO. In another aspect, the subject is administered a dose of the composition of about 10 mg/kg to about 1000 mg/kg. In another aspect, the composition is administered to the subject over a period of about 1 week to about 12 months. In another aspect, the composition is administered to the subject once daily, twice daily, once every other day, once weekly, twice weekly, once biweekly, once monthly, twice monthly, once every two or three months, or combinations thereof. In another aspect, administering the composition to the subject comprises intramuscular administration, subcutaneous administration, intravenous administration, intrathecal administration, intraperitoneal administration, or combinations thereof. In another aspect, the method further comprises performing one or more mechanical allodynia tests, heightened sensitivity to touch tests, mechanical pain sensitivity tests, or quantitative sensory tests to assess the tactile allodynia in the subject. In another aspect, the one or more mechanical allodynia tests, heightened sensitivity to touch tests, mechanical pain sensitivity tests, or quantitative sensory tests comprise von Frey filament testing, brush-evoked dynamic mechanical allodynia testing, cotton swab or light touch stimulation testing, pinprick or punctate mechanical stimulation testing, mechanical withdrawal threshold testing, mechanical pain threshold testing, quantitative sensory testing assessing mechanical detection threshold, mechanical pain threshold, mechanical pain sensitivity, or dynamic mechanical allodynia, or combinations thereof. In another aspect, the method increases proliferation of microglia cells in the subject relative to before the subject is administered the composition. In another aspect, the method increases one or more of histone H3 acetylation (H3ac) or histone H3 lysine 4 trimethylation (H3K4me3) in brain cells of the subject relative to before the subject is administered the composition. In another aspect, the method improves one or more of cognition, memory, sensory responses, or learning in the subject relative to before the subject is administered the composition. In another aspect, the method does not affect post-natal hippocampal neurogenesis in the subject.

Another embodiment described herein is a method of improving one or more of cognition, memory, sensory responses, or learning in a subject having Kabuki Syndrome, the method comprising administering to the subject a therapeutically effective amount of a composition comprising a histone deacetylase inhibitor, a polyethylene glycol (PEG), a cyclodextrin, and dimethyl sulfoxide (DMSO), thereby improving one or more of cognition, memory, sensory responses, or learning in the subject. In one aspect, the composition comprises vorinostat, PEG-400, 2-hydroxypropyl-β-cyclodextrin (HP-β-CD), and DMSO. In another aspect, the method further comprises performing one or more standardized neuropsychological tests to assess one or more of cognition, memory, sensory responses, or learning in the subject.

Another embodiment described herein is a kit for treating tactile allodynia in a subject having Kabuki Syndrome, the kit comprising: a composition comprising a histone deacetylase inhibitor, a polyethylene glycol (PEG), a cyclodextrin, and dimethyl sulfoxide (DMSO); optionally, administration materials for delivery to a subject; optionally, buffers and receptacles; and optionally, one or more of packaging, a label, or instructions for use.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-I show the effects of Kbk genotype on brain transcript levels, chromatin mark, post-natal hippocampal neurogenesis, astrocytes and microglia. FIG. 1A shows a comparison of transcript and the chromatin mark H3K4me3 illustrating SET domain in one copy of the Kmt2d gene (coding region after exon 50) replaced with the βGeo cassette in the Kbk mouse model. FIG. 1B shows qPCR analysis of brain showing 50% of Kmt2d transcripts (measured by detection of exon 52) lacked the SET domain in Kbk relative to WT mice; n=9-10 (4-5 F, 5 M) per group. FIG. 1C shows Western blots of histone H3 lysine 4 trimethylation (H3K4me3) levels in the brain of Kbk mice. The lower panel shows equivalent sample loading shown by Coomassie brilliant blue (CBB). FIG. 1D shows post-natal neurogenesis with micrographs show doublecortin (DCX, green) labeled neuroblasts in the SGZ of the hippocampal DG of WT and Kbk mice, as detected by indirect immunofluorescence assays. Mutants were un-injected (un) or injected with Vo (in DMSO+PEG) or TCF. Images shown are representative of 4-6 mice (2-3 F, 2-3 M, 2 months) per group. Blue indicates 4′,6-diamidino-2-phenylindole (DAPI) stain. Scale bar, 25 μm. FIG. 1E shows quantification of DCX positive cells along the entire SGZ Brain sections (5 mm thick spaced at 40 mm) at 4-6 planes per mouse were analyzed; n=4-6 mice/group. Statistical analysis was conducted by one-way ANOVA with Tukey's posthoc test. FIG. 1F shows micrographs illustrating GFAP positive astrocytes (brown) in hippocampi, as detected by immunohistochemistry. FIG. 1G shows quantitation of hippocampal astrocytes after excluding granule cell regions and polymorph layer of the DG (to eliminate GFAP expressing neural progenitors). Percent GFAP positive area was calculated in 4-5 random fields at three depths (consistent across all mice) and the value from each image was used to plot the data. Statistical analysis by Mann-Whitney test. FIG. 1H shows micrographs illustrating IBA1 positive microglia (brown) in the hippocampus. FIG. 1I shows quantitative analyses of hippocampal IBA1 positive microglia (under different conditions) are represented by Violin plots of images collected at 6-7 consistent planes (through the entire depth of the hippocampus). Statistical analysis was conducted by one-way ANOVA with Tukey's posthoc test.

FIG. 2A-D show single cell RNAseq analyses of gene expression changes seen in microglia isolated from mice hippocampus of WT and Kbk mice. FIG. 2A shows a graphic representation of overall design of single cell RNAseq experiments generated using BioRender. Briefly a total of 60 mice (WT and Kbk) were used. FIG. 2B shows details of samples, sequencing methods and downstream bioinformatic analyses. FIG. 2C shows UMAP of 25165 cells which are clustered into ten different sub-cell types. FIG. 2D shows the UMAP represents 12,216 microglia cells that could be computationally clustered into seven different microglial subsets (0-MG to 6-MG).

FIG. 3A-J show comparative change in microglial gene expression, predicted pathways and cellular phenotypes in Kbk and WT, dependent on age and/or TCF treatment. FIG. 3A-E show 3-month untreated mice. FIG. 3A shows differential expression of genes (DEG) in Kbk versus WT mice, with p-value <0.05; asterisks indicate fdr <0.05. FIG. 3B shows a bar plot of top 6 pathways predicted to be down regulated (blue) in Kbk by Ingenuity Pathway Analysis (IPA). FIG. 3C shows DEG of top 10 genes from FIG. 3A (red upregulated in Kbk versus WT). FIG. 3D shows IHC of Iba1 stained microglia and FIG. 3E shows the associated quantification of soma intensity and size by Imaris. FIG. 3F shows DEG with p-value <0.05: asterisks indicate fdr <0.05. FIG. 3G shows the top 6 pathways predicted to be downregulated in Kbk vs WT by IPA. FIG. 3H-J show the effects of TCF-treatment versus vehicle control (HP-β-CD) administered once weekly for three months. FIG. 3H-I show DEGs with p-value <0.05: asterisks indicate fdr <0.05. FIG. 3J shows a bar plot of top 10 pathways predicted by IPA based on DEG responses in FIG. 3H-I. In FIG. 3A-C and FIG. 3F-J, red and blue respectively show relative up and downregulation of genes or pathways.

FIG. 4A-D show TCF targets Histone H3 dependent increase of open chromatin independent of Kmt2d gene as predicted by IPA and validated in mouse brains. FIG. 4A shows Ingenuity Pathway Analysis (IPA) analyses of DEG shown in FIG. 3J, suggest that in Kbk mice, weekly TCF administered over 3 months, targets a single histone H3 (red; p<0.05), acetylation (H3Ac) of which is predicted to stimulate opening of chromatin (orange) independent of Kmt2D (black box). Elevation of H3 may reflect increased life span of the S phase the time of greatest histone transcription64. FIG. 4B-C show validation of TCF-mediated H3Ac and H3K4me3 in whole brain. Quantitative analysis of western blots showing fold change (FC) in H3Ac (FIG. 4B) or H3K4me3 (FIG. 4C). Kbk mice were given or its components as indicated, starting at P21-P23, and the brain was analyzed at ~3 months of age. All data were normalized for total histone protein and fold change is relative to the average of wild type (WT) mice. n=10 (5F, 5M)/experimental group. FIG. 4D shows correlative analyses of levels of H3Ac and H3K4me3 in brains of 50 mice. Pearson correlation co-efficient (r)=0.7, P<0.0001. Data are separated by gender (males, blue; female red). Pearson correlation co-efficient (r) males, r=0.8, P<0.0001; females, r=0.5, P=0.008. Un, untreated. P values by unpaired, two-tailed Student's t-test. *P <0.05, **P<0.001, ****P<0.0001.

FIG. 5A-C show cell trajectory analyses using pseudotime analyses tracking homeostatic and disease markers. FIG. 5A shows a UMAP plot illustrating the dimensionality reduction of microglia derived from a scRNA-seq analysis of 12,216 cells. Each cell is represented as a point in the plot, with colors corresponding to their pseudotime values. The smooth color gradient across the UMAP indicates a progression of cellular states. FIG. 5B shows a scatter plot illustrating the distribution of microglia cells across 25 equal intervals (bins) along the trajectory. The solid line represents the linear regression fit, indicating the relationship between cell proportion and trajectory. The shaded gray area surrounding the line represents the 95% confidence interval (CI), giving a visual of the statistical certainty of the regression line. The Pearson correlation coefficient (R) and p-value from a two-sided statistical test are displayed, indicating the strength and significance of the correlation between the variables. FIG. 5C shows a scatter plot illustrating the correlation between module scores and pseudotime (trajectory) for different gene modules, specifically the Homeostatic (Hexb, Tmem119, Cx3cr1, P2ry12, P2ry13, Cst3, Cd33, Csf1r, Ctss, Tmsb4x, and C1qb), Stage 1 DAM (Tyrobp, Ctsb, Apoe, B2m, Fth1, Trem2, and Ctsd) and Stage 2 DAM (Lpl, Cst7, Axl, Itgax, Spp1, Cd9, Ccl6, Csf1, Trem2, Timp2 and Ctsl) gene modules. The solid lines show less regression for each gene signature, and the shaded gray areas around them represent the 95% confidence intervals (CIs). These regressions help visualize how the module scores change across the cell trajectory.

FIG. 6A-D show defects in microglia in the hippocampus of KS mice and their response to TCF. FIG. 6A shows micrographs illustrating density of IBA1 positive microglia (black) in the hippocampus. Treatments are as indicated. Representative images collected at 6-7 consistent depths (through the entire depth of the hippocampus) with 40× objective are shown. FIG. 6B shows violin plots of Imaris-based quantitative data of microglial filament length, number of branch and terminal points assessed from 400-450 hippocampal microglia (40±10 per mouse) per group. FIG. 6C shows violin plots of microglia soma size and circularity, and FIG. 6D shows mean IBA1 intensity, as assessed from 600-700 (60±15 per mouse) microglia per group. For FIG. 6B-D, microglia were taken from two random fields at two consistent depths of the hippocampus in each mouse. Statistical analysis in FIG. 6B-D was undertaken by nonparametric Kruskal-Wallis test. In all panels, n=8-9 mice (4-5 females, 4 males) was used per group at 3 months of age. WT wild type, Kbk Kabuki, Un Untreated, HP-β-CD 2-hydroxypropyl-β-cyclodextrin, TCF triple combination formulation. **P<0.01, ***P<0.001, ****P<0.0001.

FIG. 7 shows Imaris renditions of IBA1 stained microglia: effect TCF on surveillance phenotypes in hippocampus of Kbk mice. Rendered Imaris models showing extensions and soma in hippocampal microglia in wild type mice and Kmt2d^{+/βGeo} untreated or treated with TCF or its components (as indicated). Representative models of microglia (based on 200-300 microglia per group, ~40-75 per mouse) from males, show characteristics of branched and long extensions (90-100 percentile, high), intermediate size branches and ramification (median) and small filaments with fewer branches (1-10 percentile; low). Females show the same trends (not shown) but representative models of microglia from TCF-treated females are shown for comparison. IBA1-stained images of brain sections were taken from two random fields at two different depths in each mouse (n=8-9, 4-5 females, 4 males; ages 3 months). WT wild type, Kbk Kabuki, Un Untreated, HP-β-CD 2-hydroxypropyl-β-cyclodextrin, TCF triple combination formulation. Scale bar, 7 μm.

FIG. 8A-E show TCF-induced behavioral responses and a model H3K4m3-microglial differentiation without activation in the Kabuki hippocampus. FIG. 8A shows discrimination index (DI) was determined using the NOR assay to assess the hippocampal function of learning and memory of Kbk mice treated with TCF or its components as indicated. Mice were assessed at 5-7 months of age. Numbers of mice in different groups are WT, n=25 (12F, 13M), Kbk left un-injected (un, n=24, 10F, 14M) or injected with DMSO (n=10, 5F, 5M), PEG with DMSO (n=12, 6F, 6M), HP-β-CD in PEG and DMSO (n=20, 11F, 9M) or TCF (n=18, 9F, 9M). Data are mean±SEM, one-way analysis of variance (ANOVA) with Tukey's posthoc test. FIG. 8B shows a bar diagram showing paw withdrawal threshold, an indicator of tactile sensitivity assessed by the Von Frey (VF) assay. Both the left and right hind paws of each mouse were assessed on three consecutive days and average values were used for plotting. Numbers of mice and ages were, WT, n=41, 19F, 22M, 3-7 months), Kbk left un-injected (un, n=38, 17F, 21M, 3-7 months) or injected with DMSO (n=26, 10F, 16M, 3-7 months), PEG with DMSO (n=24, 12F, 12M, 3-7 months), Vo in PEG and DMSO (n=14, 7F, 7M, 3-4 months), HP-β-CD in PEG and DMSO (n=47, 21F, 26M, 3-7 months), or TCF (n=51, 25F, 26M, 3-7 months). Data are mean±SEM, nonparametric Kruskal-Wallis test. FIG. 8C shows a model for TCF-induced H3K4m3-microglial differentiation without activation in Kbk mice. In most neurodegenerative disorders, homeostatic microglia in response to increasing the open chromatin mark H3K4me3, differentiate and become activated to express elevated levels of Iba1 and other disease-associated microglia markers (DAMs). Activated microglia further transform into reactive microglia that display high levels inflammatory secretions and neurotoxicity. FIG. 8D shows Kbk microglia, which are neither in activated nor surveillance state, undergo post-natal reduction of inflammatory markers and near global shut down of transcription from 1-3 months. TCF and H3-mediated increase of H3K4me3 open chromatin mark induced microglial differentiation, but not activation, rather differentiated microglia persist in a non-diseased surveillance state which confers behavioral benefit in mice. FIG. 8E shows in response to TCF, wild type homeostatic microglia do not undergo differentiation, but remain as non-diseased surveillance types with mild transcriptional suppression, suggesting high levels of heterochromatin protects against TCF-induced activation and differentiation and this may also limit activation of mutants with restored chromatin shown in FIG. 8D.

FIG. 9A-D show the effects of sex on the molecular and cellular characteristics of a Kbk mutant brain as compared to a wild type brain. FIG. 9A shows qPCR analysis of Kmt2d transcripts (measured by detection of exon 52) in male (M) and female (F) Kbk mice compared to wild type (WT) counterparts. Data are mean±SEM. FIG. 9B shows quantification of DCX (doublecortin X) positive neuroblasts in the entire subgranular zone (SGZ) of the dentate gyrus (DG, measured over the entire depth of the hippocampus; 5-6 sections) of WT or Kbk mutants either untreated (Un) or treated with vorinostat (Vo) or TCF; n=4-6 mice/group. Statistical analysis was conducted by one-way ANOVA with Tukey's posthoc test. FIG. 9C shows quantitative analysis of GFAP-labeled astrocytes in the hippocampus from WT and Kbk mice. Granule cell regions and the polymorph layer of the DG were excluded to eliminate neural progenitors (that are also labeled with GFAP). Percentage GFAP positive areas calculated in 4-5 random fields at three depths (across all mice) are shown. Statistical analysis was conducted by Mann-Whitney test. FIG. 9D shows violin plots showing quantification of IBA1 positive microglia in hippocampus after no treatment (Un), treatment with vorinostat (Vo) or TCF. Statistical analyses used one-way ANOVA with Tukey's post hoc test. ns, non-significant. *P<0.05, ***P<0.001, ***P<0.0001.

FIG. 10A shows a summary of the experimental design of single cell RNA sequencing of wild type and mutant microglia and their responses to chronic TCF and its components. Twenty-four Kabuki (Kbk) and 24 wild type (WT) mice at one month of age (equal numbers of males and females) were separated into four sets of 6 (3 males, 3 females). For both Kbk and WT groups, one set was left un-injected, while the remaining three sets received weekly injections of TCF or control treatments of DMSO+PEG or HP-β-CD+DMSO+PEG, up to three months of age. For subsequent analyses, to both Kbk and WT groups, another set of six untreated (Un) mice (3 males, 3 females) were added at one month of age. Parameters of treatment, age, and gender were randomized to minimize batch-differences. Hippocampi were purified and subsequently isolated cells were processed through the indicated single cell pipeline where the batch-quality was closely monitored to eliminate batch-effects (see FIG. 10B-D). Canonical microglial markers and proportion graphs were obtained as described in FIG. 10E-F. FIG. 10B shows an overview of the main steps of single-cell RNA sequencing analysis. Isolated cells were hash-tagged to differentiate between Kbk and WT, and underwent single cell capture (by barcoded beads), library preparation, RNA seq, cell ranger for seq assembly and Seurat analyses. FIG. 10C shows the preprocessing steps undertaken after single-cell RNA sequencing. FASTQ sequences were inputted into the Cell Ranger pipeline, which processed them by aligning the reads to a reference genome and generated count data for each gene. FIG. 10D shows quality control of single cell analyses. Removal of low-quality information was done using Seurat in the following manner. Cells with fewer than 500 detected genes (nFeature_RNA), more than 10% mitochondrial gene expression, and transcript counts (nCount_RNA) exceeding 30000, were excluded. Distribution of genes (nFeature_RNA), gene counts (nCount_RNA), and mitochondrial percentage (percent.mt) are shown in the top two rows across WT and Kbk genotypes before and after filtering for low quality cells. Distribution of genes (nFeature_RNA), gene counts (nCount_RNA), and mitochondrial percentage (percent.mt) are shown in the bottom two rows across batches before and after filtration of low quality cells. FIG. 10E shows a dot plot representation of canonical microglial markers. Four microglial clusters (MG1, MG2, MG3, and MG4) were enriched for 11 canonical markers, as shown. FIG. 10F shows proportion maps. Data were extracted from original cell type clusters and re-clustered using R package Seurat. The proportion of each of the microglial subsets was calculated and visualized using ggplot2 package.

FIG. 11A-C show differential gene expression in hippocampi of Kbk mice compared to wild type (WT) mice under different conditions. FIG. 11A shows differential gene expression (determined using DESeq2 package of R) induced by HP-β-CD (in DMSO+PEG) compared to PEG (in DMSO) at 3 months in Kbk mice. Genes with p-value threshold of 0.05 were filtered and used for the heat map. Asterisks correspond to genes filtered using padj (p-adjusted)<0.05. FIG. 11B shows the top 30 pathways changed in Kbk versus WT mice at 3 months of age, as predicted by IPA. FIG. 11C shows the top 30 pathways changed in Kbk versus WT mice at one month of age, as predicted by IPA.

FIG. 12 shows a magnification of FIG. 4A.

FIG. 13A-B show the effects of sex on TCF-induced increase in histone H3K4 trimethylation and acetylation in mouse brain. FIG. 13A-B show quantitative analysis of western blots showing fold change (FC) of H3K4me3 and histone 3 acetylation (H3Ac) in the brain after the treatments. Kbk mice were given TCF or its components as indicated, starting at P21-P23, and the brain was analyzed at 3 months of age. WT mice were untreated. All data were normalized for total histone protein and fold change is relative to the average of WT mice. Each group consisted of 5 males or 5 females. Data are mean±SEM.

FIG. 14A-B show quantitative morphometric analysis of hippocampal microglia in male and female Kbk mice. FIG. 14A-B show violin plots showing quantitative results of microglia filaments (length, branch, and terminal points) and soma (size, circularity, and IBA1 intensity) in untreated and treated males (FIG. 14A) and females (FIG. 14B). Treatments were as indicated. Approximately 200-350 microglia (~30-75 per mouse) from each gender were analyzed. Microglia were imaged in two random fields at two consistent depths of the hippocampus in each mouse. Males, n=4; females, n=4-5, analyzed at 3 months of age. WT, wild type; Un, untreated; TCF, triple combination formulation. Statistical analysis was conducted by Kruskal-Wallis test. ns, non-significant. *P<0.05, **P<0.01, ****P<0.0001.

FIG. 15A-B show sex-based evaluation of memory and tactile responses in drug-treated Kbk mice. FIG. 15A shows bar diagrams showing discrimination index determined by novel object recognition assay in females and males. FIG. 15B shows paw withdrawal threshold, an indicator of tactile sensitivity assessed by the Von Frey assay in female and male mice. Kbk animals were left untreated (Un) or given weekly i.p injections of TCF or its components as indicated starting at P21-P23 and assessed at 5-7 months of age. WT mice served as healthy control. Number of mice are as follows: WT males (n=13), females (n=12); untreated Kbk males (n=14), females (n=10); DMSO males (n=5), females (n=5); PEG in DMSO, males (n=6), females (n=6); HP-β-CD in PEG and DMSO, males (n=9), females (n=11); and TCF males (n=9), females (n=9). Data are mean±SEM. Statistical analysis was conducted by Student's t-test. ns, non-significant. *P<0.05, **P<0.01, ***P<0.0001.

### DETAILED DESCRIPTION

The present disclosure relates generally to compositions and methods for treating tactile allodynia and improving cognition, memory, sensory responses, and learning in subjects having Kabuki Syndrome. Kabuki Syndrome is a multi-system disorder associated with mutations in the KMT2D gene, which encodes a histone methyltransferase responsible for maintaining open chromatin marks, particularly histone H3 lysine 4 trimethylation (H3K4me3). Subjects with Kabuki Syndrome may exhibit varying degrees of intellectual disability, cardiac and immunological conditions, defects in sensory processing, and defects in hippocampal function. Tactile allodynia is a heightened sensory response to mechanical stimuli and represents one manifestation of hippocampal dysfunction in Kabuki Syndrome. This disclosure is based, in part, on the discovery that microglia in the hippocampus are affected in Kabuki Syndrome and may serve as therapeutic targets. In various aspects, the compositions and methods described herein do not affect post-natal hippocampal neurogenesis in subjects.

In various aspects described herein, a composition comprising a histone deacetylase inhibitor, a polyethylene glycol (PEG), a cyclodextrin, and dimethyl sulfoxide (DMSO) is provided. In certain embodiments, the histone deacetylase inhibitor may comprise vorinostat (Vo). In certain embodiments, the PEG may comprise PEG-400. In certain embodiments, the cyclodextrin may comprise 2-hydroxypropyl-β-cyclodextrin (HP-β-CD). The formulation may be administered to subjects having Kabuki Syndrome to treat tactile allodynia and to improve one or more of cognition, memory, sensory responses, or learning.

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30 °C, including all integers and endpoints within the specified range. In another aspect, “room temperature” refers a temperature of about 15-30 °C; about 20-30 °C; about 22-30 °C; about 25-30 °C; about 27-30 °C; about 15-22 °C; about 15-25 °C; about 15-27 °C; about 20-22 °C; about 20-25 °C; about 20-27 °C; about 22-25 °C; about 22-27 °C; about 25-27 °C; about 15 °C.+10%; about 20 °C±10%; about 22 °C±10%; about 25 °C.±10%; about 27 °C.±10%; ~20 °C, ~22 °C, ~25 °C, or ~27 °C, at standard atmospheric pressure.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifested.

As used herein, the term “tactile allodynia” is a neurological condition in KS patients in which pain, including severe pain, may occur with a light touch of the skin. For example, in KS patients with tactile allodynia, there is a heightened sensitivity to touch in feet, which is called “foot hyperesthesia.” When the feet are touched, there is often a jerking reaction from the patient. With tactile allodynia, foot pain and general distress are felt in response to touch stimulation. This is all part of the sensory integration disorder and dysmorphism of KS.

Described herein are therapeutic compositions that comprise a histone deacetylase inhibitor formulated with one or more excipients and other components to facilitate delivery and bioavailability. In some cases, the histone deacetylase inhibitor may comprise vorinostat, belinostat, romidepsin, dacinostat, panobinostat, givinostat, pyroxamide, trichostatin A, m-carboxycinnamic acid bis-hydroxamide (CBHA), or combinations thereof. In some embodiments, the histone deacetylase inhibitor may comprise vorinostat (Vo). Vorinostat is a pan-histone deacetylase inhibitor that may block deacetylation of histones by targeting multiple HDACs. In some aspects, vorinostat may preferentially inhibit HDAC3 in microglia, leading to selective increases in histone H3 levels.

In various aspects, the compositions further comprise a polyethylene glycol (PEG) component, which may serve as a solubilizing agent and may enhance the delivery characteristics of the composition. The average molecular weight of the PEG may range from about 100 to 6000 Da. This range includes all values and subranges therebetween, including 100, 200, 300, 400, 500, 600, 700, 800, 900, 1000, 1200, 1400, 1600, 1800, 2000, 2100, 2200, 2300, 2400, 2500, 2600, 2700, 2800, 2900, 3000, 3500, 4000, 5000, and 6000 Da, or any combination thereof. In some embodiments, polyethylene glycol having an average molecular weight of 100-1000 Da is used. In some embodiments, polyethylene glycol having an average molecular weight of 200-600 is used. In some embodiments, polyethylene glycol having an average molecular weight of 400 is used (i.e., PEG-400). Mixtures of PEGs having different molecular weights are also contemplated. In some embodiments, the PEG may have an average molecular weight of about 200 Da to about 600 Da. In some embodiments, the PEG may comprise PEG-400, which refers to polyethylene glycol having an average molecular weight of approximately 400 Da.

In various aspects, the compositions further comprise a cyclodextrin (CD) component. Cyclodextrins are cyclic oligosaccharides that may function as solubilizing agents and may enhance the stability and bioavailability of active pharmaceutical ingredients. The cyclodextrin may have any average molecular weight ranging from about 970 to 6000 Da depending on, for example, the type of cyclodextrin (a, B, or v) and whether it is crosslinked or uncrosslinked, substituted or unsubstituted, the degree of substitution, and the like, as is known in the art.

Accordingly, the cyclodextrin may be crosslinked or uncrosslinked, substituted or unsubstituted, or any combination thereof. Referring to the molecular weight, the aforementioned range includes all values and subranges therebetween, including about 970, 972, 980, 990, 1000, 1010, 1030, 1050, 1070, 1090, 1100, 1120, 1140, 1160, 1180, 1200, 1250, 1300, 1350, 1370, 1380, 1390, 1395, 1400, 1410, 1420, 1430, 1440, 1460, 1480, 1500, 1600, 1800, 2000, 2500, 3000, 3500, 4000, 5000, and 6000 Da, or combination thereof. If substituted, the cyclodextrin may have a degree of substitution, or average number of substituents per glucopyranose unit, ranging from 0.5 to 3. This range includes any value or subrange therebetween, including 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.2, 2.4, 2.5, 2.6, 2.7, 2.8, 2.9, 3, or any combination thereof. The cyclodextrin is preferably water soluble. The cyclodextrin may have a water solubility at 25 °C of about 10 mg/ml or greater. This range includes all values and subranges therebetween, including about 10, 20, 40, 60, 100, 200, 300, 400, 500, 600 mg/mL, or greater. Mixtures of different cyclodextrins are also contemplated. In some embodiments, the cyclodextrin may be administered in an amount ranging from 1000-40,000 mg/kg. This range includes all values and subranges therebetween, including 1000, 1200, 1400, 1600, 1800, 2000, 2100, 2200, 2300, 2400, 2500, 2600, 2700, 2800, 2900, 3000, 3500, 4000, 5000, 6000, 7000, 8000, 9000, 10,000, 20,000, 30,000, 40,000 mg/kg, or any combination thereof. In some embodiments, the dosage amount is based on a murine dose, and may be scaled for human treatment, as is known in the art. In some embodiments, the cyclodextrin may have an average molecular weight of about 900 Da to about 1500 Da. In some embodiments, the cyclodextrin may comprise 2-hydroxypropyl-β-cyclodextrin (HP-β-CD), dimethyl-β-cyclodextrin, hydroxypropyl-α-cyclodextrin, hydroxypropyl-γ-cyclodextrin, or combinations thereof. In some embodiments, the cyclodextrin may comprise 2-hydroxypropyl-β-cyclodextrin (HP-β-CD).

In various aspects, the compositions further comprise dimethyl sulfoxide (DMSO). DMSO may function as a solvent and pharmaceutically acceptable carrier and may enhance the permeability of the composition across biological membranes. Thus, DMSO may act as a penetration enhancer.

In various aspects, the composition may comprise about 10 wt % to about 40 wt % histone deacetylase inhibitor, about 15 wt % to about 50 wt % PEG, about 20 wt % to about 50 wt % cyclodextrin, and about 5 wt % to about 10 wt % DMSO.

In various aspects, the histone deacetylase inhibitor is present in an amount of about 10 wt % to about 40 wt % based on the total weight of the composition. In further aspects, the histone deacetylase inhibitor may be present in an amount of about 10 wt % to about 38 wt %, about 10 wt % to about 35 wt %, about 10 wt % to about 32 wt %, or about 10 wt % to about 30 wt %. In other aspects, the histone deacetylase inhibitor may be present in an amount of about 12 wt % to about 40 wt %, about 15 wt % to about 40 wt %, about 18 wt % to about 40 wt %, or about 20 wt % to about 40 wt %. In some embodiments, the histone deacetylase inhibitor is present in an amount of about 12 wt % to about 35 wt %, about 15 wt % to about 30 wt %, about 18 wt % to about 28 wt %, or about 20 wt % to about 25 wt %. In particular embodiments, the histone deacetylase inhibitor may be present in an amount of about 10 wt %, 12 wt %, 15 wt %, 18 wt %, 20 wt %, 22 wt %, 25 wt %, 28 wt %, 30 wt %, 32 wt %, 35 wt %, 38 wt %, or 40 wt %.

In various aspects, PEG is present in an amount of about 15 wt % to about 50 wt % based on the total weight of the composition. In further aspects, PEG may be present in an amount of about 15 wt % to about 48 wt %, about 15 wt % to about 45 wt %, about 15 wt % to about 42 wt %, or about 15 wt % to about 40 wt %. In other aspects, PEG may be present in an amount of about 18 wt % to about 50 wt %, about 20 wt % to about 50 wt %, about 25 wt % to about 50 wt %, or about 30 wt % to about 50 wt %. In some embodiments, PEG is present in an amount of from about 20 wt % to about 45 wt %, from about 25 wt % to about 40 wt %, from about 28 wt % to about 38 wt %, or from about 30 wt % to about 35 wt %. In particular embodiments, PEG may be present in an amount of about 15 wt %, 18 wt %, 20 wt %, 25 wt %, 30 wt %, 35 wt %, 40 wt %, 42 wt %, 45 wt %, 48 wt %, or 50 wt %.

In various aspects, the cyclodextrin is present in an amount of about 20 wt % to about 50 wt % based on the total weight of the composition. In further aspects, the cyclodextrin may be present in an amount of about 20 wt % to about 48 wt %, about 20 wt % to about 45 wt %, about 20 wt % to about 42 wt %, or about 20 wt % to about 40 wt %. In other aspects, the cyclodextrin may be present in an amount of about 22 wt % to about 50 wt %, about 25 wt % to about 50 wt %, about 30 wt % to about 50 wt %, or about 35 wt % to about 50 wt %. In some embodiments, the cyclodextrin is present in an amount of about 25 wt % to about 45 wt %, about 30 wt % to about 40 wt %, about 32 wt % to about 38 wt %, or about 34 wt % to about 36 wt %. In particular embodiments, the cyclodextrin may be present in an amount of about 20 wt %, 22 wt %, 25 wt %, 30 wt %, 32 wt %, 35 wt %, 38 wt %, 40 wt %, 45 wt %, 48 wt %, or 50 wt %.

In various aspects, DMSO is present in an amount of about 5 wt % to about 10 wt % based on the total weight of the composition. In further aspects, DMSO may be present in an amount of about 5 wt % to about 9.5 wt %, about 5 wt % to about 9 wt %, about 5 wt % to about 8.5 wt %, or about 5 wt % to about 8 wt %. In other aspects, DMSO may be present in an amount of about 5.5 wt % to about 10 wt %, about 6 wt % to about 10 wt %, about 6.5 wt % to about 10 wt %, or about 7 wt % to about 10 wt %. In some embodiments, DMSO is present in an amount of about 6 wt % to about 9 wt %, about 6.5 wt % to about 8.5 wt %, about 7 wt % to about 8 wt %, or about 7.2 wt % to about 7.8 wt %. In particular embodiments, DMSO may be present in an amount of about 5 wt %, 5.5 wt %, 6 wt %, 6.5 wt %, 7 wt %, 7.5 wt %, 8 wt %, 8.5 wt %, 9 wt %, 9.5 wt %, or 10 wt %.

In various aspects, the composition may comprise a triple combination formulation (TCF) as disclosed herein. The TCF comprises vorinostat, PEG-400, 2-hydroxypropyl-β-cyclodextrin (HP-β-CD), and DMSO, each present in an amount effective to provide the desired solubility, stability, and bioavailability characteristics of the composition. In certain nonlimiting exemplary embodiments, the triple combination formulation may comprise vorinostat at a dose of about 50 mg/kg, HP-β-CD at a dose of about 2000 mg/kg, PEG 400 at about 45 wt %, and DMSO at about 5 wt %. The TCF may be brain permeant and may be well tolerated in long term administration.

Also described herein are methods of treating tactile allodynia in subjects having Kabuki Syndrome using the composition disclosed herein. Tactile allodynia refers to heightened sensitivity to mechanical stimuli that are normally non-painful. In subjects with Kabuki Syndrome, tactile hypersensitivity may manifest as tactile avoidance behaviors in response to mechanical stimulation. The methods described herein may improve tactile responses in subjects having Kabuki Syndrome by administering a composition comprising a histone deacetylase inhibitor, a polyethylene glycol (PEG), a cyclodextrin, and dimethyl sulfoxide (DMSO). The methods may improve one or more of cognition, memory, sensory responses, or learning in the subject relative to before the subject is administered the composition.

In various aspects, the composition may be administered to the subject over a period of about 1 week to about 12 months. The composition may be administered to the subject once daily, twice daily, once every other day, once weekly, twice weekly, once biweekly, once monthly, twice monthly, once every two or three months, or combinations thereof. In certain embodiments, the composition is administered once weekly. Weekly administration may enable short exposure of the histone deacetylase inhibitor with intermittent rest periods, which may be beneficial for selective action through histone H3. In some aspects, administering the composition to the subject may comprise intramuscular administration, subcutaneous administration, intravenous administration, intrathecal administration, intraperitoneal administration, or combinations thereof. The subject may be administered a dose of the composition of about 10 mg/kg to about 1000 mg/kg. In some aspects, the injection volume of the composition may be about 10 mL/kg.

In some aspects, the methods may further comprise performing one or more mechanical allodynia tests, heightened sensitivity to touch tests, mechanical pain sensitivity tests, or quantitative sensory tests to assess the tactile allodynia and any improvements in the subject. Such assessments may be conducted at baseline and at one or more subsequent time points to evaluate tactile allodynia and hypersensitivity and to determine any improvements or changes in sensory function over time or in response to administration of a composition disclosed herein. These tests are designed to assess abnormal pain responses to normally non-painful mechanical stimuli, altered mechanical pain thresholds, or exaggerated responses to tactile stimulation. Such tests may include, but are not limited to, von Frey filament testing, brush-evoked dynamic mechanical allodynia testing, cotton swab or light touch stimulation testing, pinprick or punctate mechanical stimulation testing, mechanical withdrawal threshold testing, mechanical pain threshold testing, quantitative sensory testing assessing mechanical detection threshold, mechanical pain threshold, mechanical pain sensitivity, or dynamic mechanical allodynia, or combinations thereof. In some aspects, test results may be expressed as raw scores, standardized scores, age-adjusted scores, developmental quotients, or changes from baseline, and may be compared to normative reference values or to the subject's own prior performance. In some embodiments, the compositions and methods disclosed herein may improve tactile allodynia in a subject having KS by at least about 5%, 10%, 15%, 20%, 25%, 30%, 35%, 40%, 45%, 50%, 55%, 60%, 65%, 70%, 75%, 80%, 85%, 90%, or 95% relative to before the subject is administered a composition, as assessed by one or more mechanical allodynia tests, heightened sensitivity to touch tests, mechanical pain sensitivity tests, or quantitative sensory tests disclosed herein.

In some aspects, the methods may increase the proliferation of microglia cells in the subject relative to before the subject is administered the composition. The methods may increase microglial densities in the hippocampus. In some aspects, the methods may promote one or more of microglial filament extension length, numbers of dendrite branch points, and terminal points in the subject. The methods may also increase soma size and reduce circularity of microglia in subjects, which may be associated with surveillance phenotypes rather than activated or inflammatory states.

In some aspects, the methods may increase one or more of histone H3 acetylation (H3ac) or histone H3 lysine 4 trimethylation (H3K4me3) in brain cells of the subject relative to before the subject is administered the composition. Without being bound by theory, the therapeutic effects of the compositions and methods disclosed herein may be mediated through acetylation-dependent promotion of H3K4me3 open chromatin marks. In some aspects, there may be a positive correlation between acetylation and trimethylation levels in the brain of the subject. The methods may also drive microglial differentiation toward surveillance forms without increasing disease-associated microglia markers (DAMs). In some aspects, the methods may not affect post-natal hippocampal neurogenesis in the subject. Without being bound by theory, the therapeutic effects may be mediated through effects on microglia rather than through stimulation of neurogenesis.

Also described herein are methods of improving one or more of cognition, memory, sensory responses, or learning in a subject having Kabuki Syndrome using the composition disclosed herein. Kabuki Syndrome is associated with varying degrees of intellectual disability, and subjects may exhibit deficits in hippocampal functions including learning and memory. In subjects having Kabuki Syndrome, impairments in cognition, memory, sensory processing and responses, and learning are commonly observed and represent key areas for improvement. Many KS patients exhibit mild to moderate intellectual disability, with particular challenges in working memory formation and retention, sensory integration, attention, learning capacity and executive function that can interfere with academic performance and daily activities. Sensory responses, including hypersensitivity or hyposensitivity to auditory, visual, or tactile stimuli, may further complicate learning and social engagement. The compositions and methods described herein may improve cognition, memory, sensory responses, and/or learning in subjects having Kabuki Syndrome by administering a composition comprising a histone deacetylase inhibitor, a polyethylene glycol (PEG), a cyclodextrin, and dimethyl sulfoxide (DMSO).

In some aspects, the methods further comprise performing one or more standardized neuropsychological tests to assess one or more of cognition, memory, sensory responses, or learning in the subject. In subjects having Kabuki syndrome, one or more aspects of cognition, memory, sensory responses, and learning may be assessed using standardized neuropsychological and behavioral testing techniques and instruments. In some aspects, such assessments may be performed at baseline and at one or more subsequent time points following administration of a composition disclosed herein in order to evaluate neurocognitive function, developmental progress, or changes associated with therapeutic intervention. Standardized neuropsychological tests may be selected to evaluate, for example, global cognitive ability, executive function, attention, working memory, learning capacity, or information processing speed, including age-appropriate intelligence scales, or memory assessments. In some aspects, memory and learning may be assessed using standardized tests designed to measure short-term memory, long-term memory, verbal learning, visual learning, or memory retention, such as list-learning tasks, recall and recognition paradigms, or paired-associate learning tests. Cognitive function may be further evaluated using tests of problem solving, reasoning, attention, or adaptive behavior, including caregiver- or clinician-administered questionnaires. In addition, sensory responses may be assessed using standardized sensory processing measures or behavioral assessments that evaluate responsiveness or sensitivity to auditory, visual, tactile, proprioceptive, or vestibular stimuli. In some aspects, test results may be expressed as raw scores, standardized scores, age-adjusted scores, developmental quotients, or changes from baseline, and may be compared to normative reference values or to the subject's own prior performance. Improvements in one or more test outcomes may indicate enhanced cognitive function, improved memory formation or retention, improved sensory integration, or improved learning ability in the subject. Such standardized assessments provide objective and reproducible measures for evaluating neurodevelopmental function in KS subjects and for monitoring changes over time or in response to the therapeutic treatments disclosed herein. In some embodiments, the compositions and methods disclosed herein may improve one or more of cognition, memory, sensory responses, or learning in a subject having KS by at least about 5%, 10%, 15%, 20%, 25%, 30%, 35%, 40%, 45%, 50%, 55%, 60%, 65%, 70%, 75%, 80%, 85%, 90%, or 95% relative to before the subject is administered a composition, as assessed by one or more standardized neuropsychological tests disclosed herein.

Also described herein are kits for treating tactile allodynia in a subject having Kabuki Syndrome. Kits for improving one or more of cognition, memory, sensory responses, or learning in a subject having Kabuki Syndrome are also provided. The kits comprises a composition comprising a histone deacetylase inhibitor, a polyethylene glycol (PEG), a cyclodextrin, and dimethyl sulfoxide (DMSO). In some embodiments, the disclosed kits may optionally further comprise one or more of administration materials for delivery to a subject; buffers and receptacles; or one or more of packaging, a label, or instructions for use.

The kits described herein may be designed for use in various laboratory or clinical settings. The kit may include all necessary components and detailed instructions to facilitate safe and effective use in these settings. The instructions may be tailored to the level of expertise expected of the user, with more detailed guidance provided for settings where less specialized personnel may be involved in preparation or use. The kits described herein may comprise protective packaging to ensure the integrity of the components during storage and transport. In some embodiments, the kit may comprise cushioning materials, such as foam inserts or bubble wrap, to protect glass vials or ampoules from breakage. The kit may comprise tamper-evident seals or packaging to ensure that the kit has not been opened or tampered with prior to use. Instructions included in kits may be affixed to packaging material or may be included as a package insert. While the instructions are typically written on printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. As used herein, the term “instructions” may include the address of an internet site that provides the instructions.

One embodiment described herein is a method of treating tactile allodynia in a subject having Kabuki Syndrome, the method comprising administering to the subject a therapeutically effective amount of a composition comprising a histone deacetylase inhibitor, a polyethylene glycol (PEG), a cyclodextrin, and dimethyl sulfoxide (DMSO), thereby treating the tactile allodynia in the subject. In one aspect, the histone deacetylase inhibitor comprises vorinostat, belinostat, romidepsin, dacinostat, panobinostat, givinostat, pyroxamide, trichostatin A, m-carboxycinnamic acid bis-hydroxamide (CBHA), or combinations thereof. In another aspect, the histone deacetylase inhibitor comprises vorinostat. In another aspect, the PEG has an average molecular weight of about 200 Da to about 600 Da. In another aspect, the PEG comprises PEG-400. In another aspect, the cyclodextrin has an average molecular weight of about 900 Da to about 1500 Da. In another aspect, the cyclodextrin comprises 2-hydroxypropyl-β-cyclodextrin (HP-β-CD), dimethyl-β-cyclodextrin, hydroxypropyl-α-cyclodextrin, hydroxypropyl-γ-cyclodextrin, or combinations thereof. In another aspect, the cyclodextrin comprises 2-hydroxypropyl-β-cyclodextrin (HP-β-CD). In another aspect, the composition comprises vorinostat, PEG-400, 2-hydroxypropyl-β-cyclodextrin (HP-β-CD), and DMSO. In another aspect, the composition comprises about 10 wt % to about 40 wt % histone deacetylase inhibitor; about 15 wt % to about 50 wt % PEG; about 20 wt % to about 50 wt % cyclodextrin; and about 5 wt % to about 10 wt % DMSO. In another aspect, the subject is administered a dose of the composition of about 10 mg/kg to about 1000 mg/kg. In another aspect, the composition is administered to the subject over a period of about 1 week to about 12 months. In another aspect, the composition is administered to the subject once daily, twice daily, once every other day, once weekly, twice weekly, once biweekly, once monthly, twice monthly, once every two or three months, or combinations thereof. In another aspect, administering the composition to the subject comprises intramuscular administration, subcutaneous administration, intravenous administration, intrathecal administration, intraperitoneal administration, or combinations thereof. In another aspect, the method further comprises performing one or more mechanical allodynia tests, heightened sensitivity to touch tests, mechanical pain sensitivity tests, or quantitative sensory tests to assess the tactile allodynia in the subject. In another aspect, the one or more mechanical allodynia tests, heightened sensitivity to touch tests, mechanical pain sensitivity tests, or quantitative sensory tests comprise von Frey filament testing, brush-evoked dynamic mechanical allodynia testing, cotton swab or light touch stimulation testing, pinprick or punctate mechanical stimulation testing, mechanical withdrawal threshold testing, mechanical pain threshold testing, quantitative sensory testing assessing mechanical detection threshold, mechanical pain threshold, mechanical pain sensitivity, or dynamic mechanical allodynia, or combinations thereof. In another aspect, the method increases proliferation of microglia cells in the subject relative to before the subject is administered the composition. In another aspect, the method increases one or more of histone H3 acetylation (H3ac) or histone H3 lysine 4 trimethylation (H3K4me3) in brain cells of the subject relative to before the subject is administered the composition. In another aspect, the method improves one or more of cognition, memory, sensory responses, or learning in the subject relative to before the subject is administered the composition. In another aspect, the method does not affect post-natal hippocampal neurogenesis in the subject.

Another embodiment described herein is a method of improving one or more of cognition, memory, sensory responses, or learning in a subject having Kabuki Syndrome, the method comprising administering to the subject a therapeutically effective amount of a composition comprising a histone deacetylase inhibitor, a polyethylene glycol (PEG), a cyclodextrin, and dimethyl sulfoxide (DMSO), thereby improving one or more of cognition, memory, sensory responses, or learning in the subject. In one aspect, the composition comprises vorinostat, PEG-400, 2-hydroxypropyl-β-cyclodextrin (HP-β-CD), and DMSO. In another aspect, the method further comprises performing one or more standardized neuropsychological tests to assess one or more of cognition, memory, sensory responses, or learning in the subject.

Another embodiment described herein is a kit for treating tactile allodynia in a subject having Kabuki Syndrome, the kit comprising: a composition comprising a histone deacetylase inhibitor, a polyethylene glycol (PEG), a cyclodextrin, and dimethyl sulfoxide (DMSO); optionally, administration materials for delivery to a subject; optionally, buffers and receptacles; and optionally, one or more of packaging, a label, or instructions for use.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Materials

All fine chemicals were purchased from Sigma (St Louis, MO, USA) unless otherwise indicated. Vorinostat was procured from Selleck Chemicals (Houston, TX, USA).

### Animals

Breeding pair of kabuki mouse model Kmt2d^{+/βGeo} (cataloged as Kmt2d^{Gt(RRT024)Byg}) were obtained from Bay Genomics, University of California. These mice carried a heterozygous mutation in Kmt2d gene and were on the mixed background of C57BL/6J and 129/SvEv. The Kmt2d^{+/βGeo} mice were backcrossed with WT C57BL/6J mice and strain purity (99-100%) confirmed by genetic analysis at Jackson labs. Breeding was performed in-house by pairing female wild type (WT) mice with male heterozygous mutant mice. Genotyping of the progenies was performed using PCR. Primers for the wild-type and mutant alleles are shown in Table 1. On agarose gels, one single band of size 411 bp suggested WT, whereas Kmt2d^{+/βGeo} mice produced two bands of sizes 411 bp and 272 bp.

TABLE 1
Genotyping PCR Primers

| | | SEQ |
|---|---|---|
| Name | DNA Sequence (5′→3′) | ID NO |
| WT Kmt2d Fwd | CCGAGTACCTGAAAGGCGAA | 1 |
| WT Kmt2d Rev | CTAATCAGGGCTAAGGGCGA | 2 |
| Mutant Kmt2d Fwd | GTGTGGAACCGCATCATTGAG | 3 |
| Mutant Kmt2d Rev | GTCTTTGAGCACCAGAGGACA | 4 |

### Drug Injections and Organ Harvest

The triple combination formulation (TCF) comprised a mixture of vorinostat (50 mg/kg), 2 hydroxypropyl-β-cyclodextrin (HP-β-CD, 2000 mg/kg), polyethylene glycol 400 (PEG, 45 wt %) and DMSO (5 wt %). Vehicle contained HP-β-CD (2000 mg/kg), PEG 400 (45%) and DMSO (5%). Vorinostat (50 mg/kg) was prepared in 5% DMSO and 45% PEG. Detailed methodology for preparing TCF drug solutions was described by Alam et al., Nat. Sci. Rep. 8 (1): 3875 (2018). The injection volume was 10 ml/kg. Throughout the experiment, TCF or its components were administered weekly through the intraperitoneal route, post-weaning starting at 21-23 days of age. Mice were sacrificed by asphyxiation using CO₂. The brain was isolated, the hemispheres were separated, flash-frozen in liquid nitrogen and stored at −80 °C or immersion fixed in 10% neutral buffered formalin.

RNA Extraction and qPCR Analysis

Total RNA from frozen brain hemisphere was extracted using RNeasy Plus Universal kit from Qiagen and quantitative PCR (qPCR) was performed using Power SYBR Green RNA-to-CT 1-Step Kit. A 131 bp transcript region corresponding to exon 52 of Kmt2d (region deleted in one of the alleles in mutant mice) was detected using primers targeting Kmt2d Gapdh (glyceraldehyde 3-phosphate dehydrogenase) and as shown in Table 2. The relative quantification of gene expression was done by the ΔΔC_{T} method.

TABLE 2
qPCR Primers

| Name | DNA Sequence (5′→3′) | SEQ ID NO |
|---|---|---|
| Kmt2d Fwd | GCACCTTCACAGGCGAGACC | 5 |
| Kmt2d Rev | GAGGCCCTGGATACGCGAAC | 6 |
| Gapdh Fwd | TCCATGACAACTTTGGCATTG | 7 |
| Gapdh Rev | CAGTCTTCTGGGTGGCAGTGA | 8 |

### Histone Extraction and Western Blotting

Frozen brain was homogenized in Dounce homogenizer, and the histones were extracted using ‘EpiQuik Total Histone extraction kit’ (Epigentek, NY, USA) as per manufacturer's instructions. Antibodies to acetylated histone H3, K14 (Catalog #7627) and trimethylated histone H3K4 (Catalog #9727) were from Cell Signaling Technology. Appropriate HRP-conjugated secondary antibodies were from Bio-Rad (Hercules, CA, USA). Quantification of band intensity was performed using ImageJ software (NIH, MD, USA).

### Isolation of Hippocampus and Single Cell Preparations

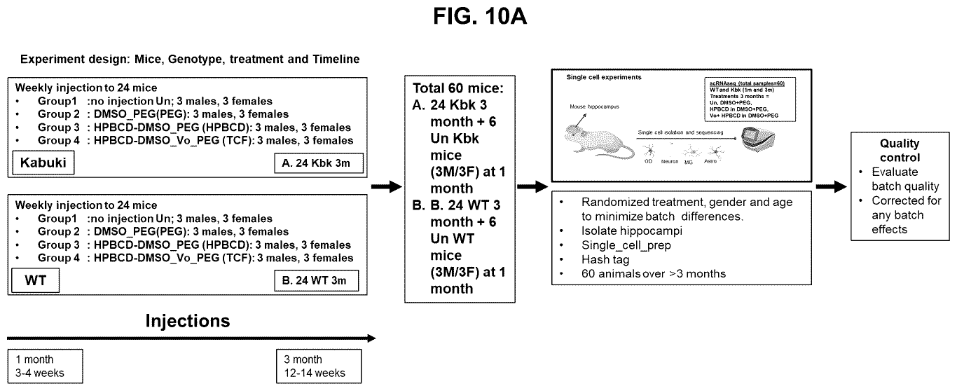

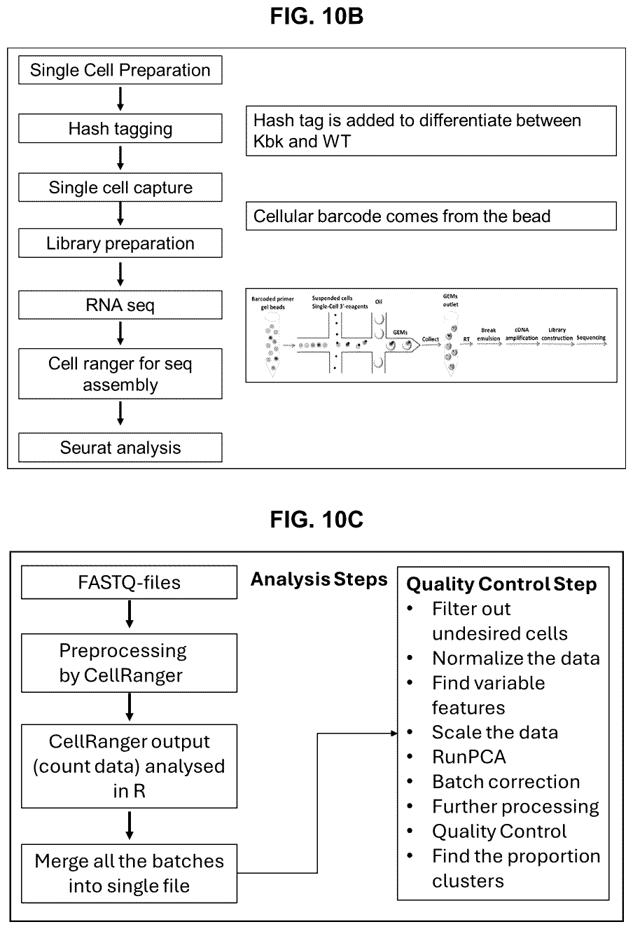

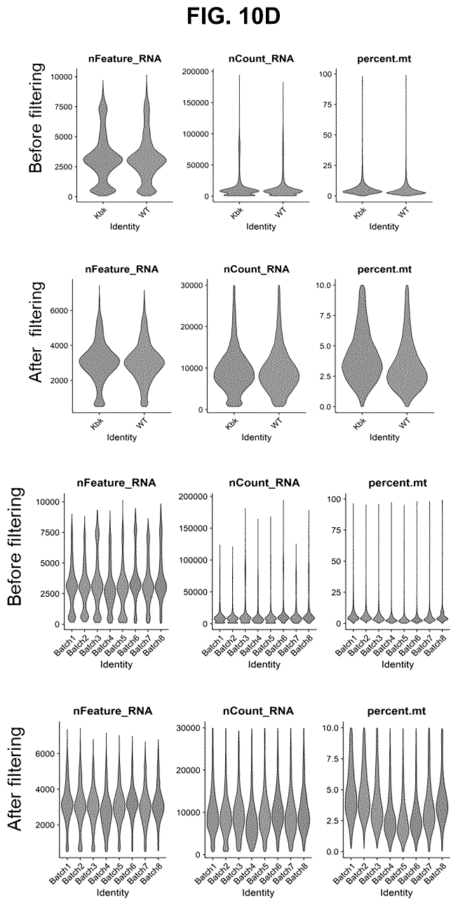

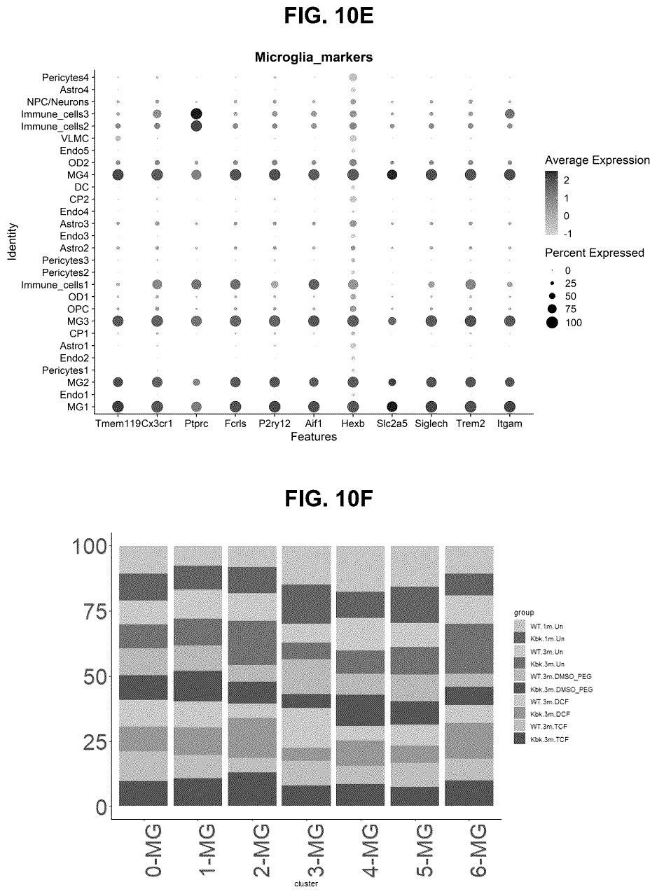

The study included a total of 60 mice, either WT or Kbk, at either one month or three-four months of age with all cohorts of mice balanced between both sexes. The one-month-old group consisted of 12 untreated mice (2 genotypes×2 genders×3 mice per gender). The 3-4-month-old group was divided into four treatment categories: untreated, PEG [dimethyl sulfoxide, 5% (DMSO) and polyethylene glycol, 45% (PEG)], HP-β-CD [DMSO (5%), PEG (45%), Hydroxypropyl-β-cyclodextrin, 0.2 μm/mL (HP-β-CD)], and TCF [DMSO (5%), PEG (45%), HP-β-CD (0.2 μm/mL), Vorinostat (5 mg/mL, Vo)]. The 3-4-month-old cohort comprised 48 mice (2 genotypes (WT and Kbk)×2 genders (males and females)×4 treatments (Untreated, PEG (+DMSO), HP-β-CD (in +DMSO+PEG), TCF)×3 mice per gender). Due to the large sample size, the experiment was conducted in batches (for more information, see FIG. 10A).

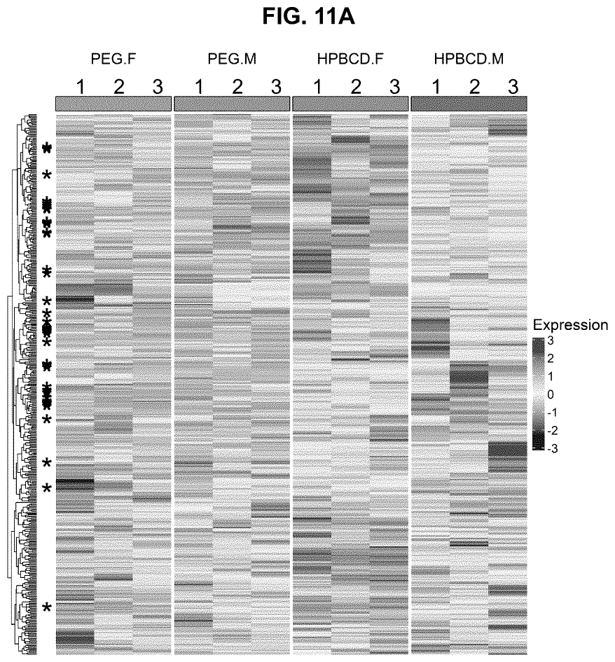

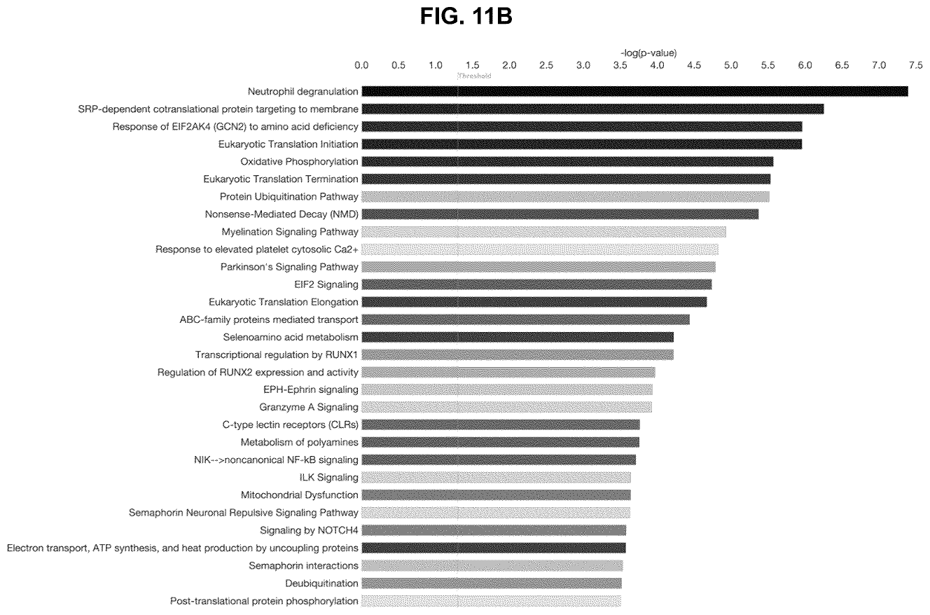

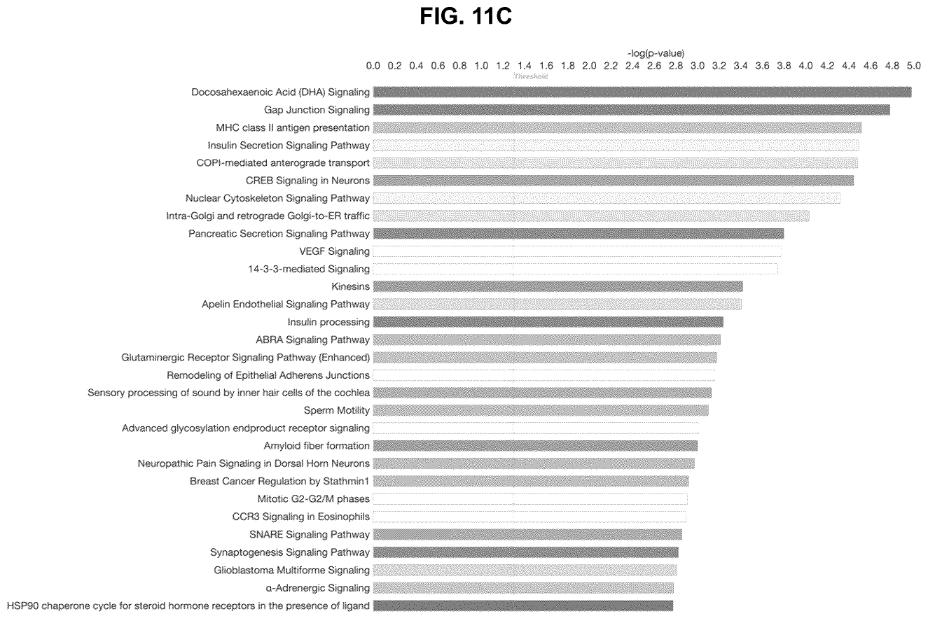

Flowcharts outlining the steps involved in single-cell isolation, processing, and analysis are summarized in FIG. 10B and FIG. 11C. Established methods were used to generate single-cell suspensions from hippocampus tissue. Briefly, mice were anesthetized with carbon dioxide and perfused using fresh, ice-cold perfusion buffer (PB+), which consisted of Hibernate A-minus Calcium (HA-Ca; BrainBits), Actinomycin D (5 μg/mL; Sigma-Aldrich; A1410), and Triptolide (10 UM; Sigma-Aldrich T3652). The brain was carefully harvested, placed in a 10 cm dish with cold PB+, and the hippocampi were dissected out and transferred into the buffer. Using a razor blade, the hippocampi were minced into fine pieces, transferred to a 15 mL tube with PB+ using a 1 mL pipette tip with blunt end, and tissue pieces were allowed to settle. The PB+ supernatant was discarded, and the minced tissue was digested in 5 mL activated papain dissolved in dissociation buffer (DXB+) supplemented with Anisomycin (27.1 μg/mL; Sigma-Aldrich; A9789) and DNase I (2 mg/mL; Roche 10104159001). Activated papain was prepared by reconstituting lyophilized papain with 5 mL DXB+, incubating at 37 °C for 10 minutes, and adding 100 μL DNase I (2 mg/mL). The minced tissue was incubated with the activated papain at 37 °C for 30 minutes with gentle shaking. Afterward, the tissue was triturated initially using 5 mL pipette followed by 1 mL pipettes (10 strokes each) until no clumps remained. An additional 200 μL DNase I (2 mg/mL) was added, and the mixture was centrifuged at 300×g for 5 minutes at RT.

The resulting pellet was resuspended in an ovomucoid solution (2.7 mL DXB+ with 300 μL reconstituted ovomucoid and 100 μL DNase I) to inactivate papain. The cells were layered onto 5 mL of ovomucoid solution in a separate tube and centrifuged at 300×g for 10 minutes. The supernatant was discarded, and the pellet was resuspended in Hibernate A-minus Calcium (HA-Ca). The suspension was passed through a pre-wetted 70 μm filter (Miltenyi) to remove debris, and the filter was rinsed with 2 mL HA-Ca to collect any remaining cells. The final cell suspension was centrifuged at 300×g for 10 minutes at 4 °C (with maximum acceleration and half brake) and the cell pellet was processed for debris removal as described below.

### Percoll Gradient for Debris Removal

The pellet was resuspended in 1 mL 30% Percoll in 1×PBS, pH 7.4, and carefully layered on 5 mL 70% Percoll in 1×PBS, pH 7.4 and another 4 mL of 30% Percoll was layered carefully on the top of cell suspension. The gradient was subjected to centrifugation for 30 minutes at 300×g at 22 °C. Debris-free single cells were collected from the 70-30% Percoll interface and transferred using a 1 mL pipette with blunted tip to another 15 mL tube containing 14 mL Hibernate A-Low Fluorescence (HA-LF) containing Actinomycin, Triptolide, and Anisomycin. The mixture was centrifuged for 10 min at 300×g, 4 °C, with maximum acceleration and brake. The cell pellet was resuspended in 300 μL of HA-LF and corresponding inhibitors.

### Cell Hashing Protocol

For cell hashing, the cells were counted using a hemocytometer. One million cells were resuspended in 100 μL of Staining buffer (2% BSA/0.02% Tween, PBS), blocked with 10 μL of Fc Blocking reagent (BioLegend), and incubated at 4 °C for 10 minutes. After blocking, 1 μg of hashing antibody was added, and the mixture was incubated for 30 minutes at 4 °C. The cells were then washed three times with Staining buffer, pelleted at 400×g for 5 minutes at 4 °C, and resuspended in 1×PBS, PH 7.4.

### Single Cell Capture, Library Preparation and Sequencing

The percent viability and concentration of the cell suspension was determined by staining Trypan Blue Stain (0.4%; Thermo Fisher Scientific PN-T10282) and Countess II Automated Cell Countess. It was loaded into Chromium Next GEM Chip G (10× Genomics PN-1000127) with a targeted recovery of 16,000 to 20,000 cells per well and processed with Chromium Controller (10× Genomics). Single-cell RNAseq libraries were prepared with Chromium Single Cell 3′ Reagent Kit (v3.1 CG000204 Rev C; 10× Genomics PN-1000128) and Chromium i7 Multiplex Kit (Single Index; PN-120262) following the standard throughput manual workflow. Single-cell HTO libraries were prepared following CITE-seq & Cell Hashing Protocol (New York Genome Center Technology Innovation Lab, version 2019 Feb. 13). The single-cell RNA-seq libraries were sequenced on an Illumina NovaSeq 6000 with paired-end reads (28 cycles Read 1, 8 cycles i7, 91 cycles Read 2).

### Single Cell Data Analysis

The Cell Ranger software (10× Genomics) was used to align the FASTQ sequencing data from the individual batches to the reference mouse transcriptome (using STAR algorithm) and the demultiplexing was performed by unique molecular identifiers (UMI) and cell specific barcodes. After demultiplexing, the number of unique molecular identifiers (UMIs) associated with each gene in each cell was quantified by Cell Ranger. The Cell Ranger output consists of three matrices where rows of feature-barcode matrix represent genes, columns represent cells, and each entry represents the expression level of a gene in a specific cell. The filtered expression matrix output of each batch was processed by R package Seurat_5.1.0. The data from each batch was (total eight batches) merged into a single Seurat object and subsequently, the single cells were subset and used for further analysis. The integrity of individual cells obtained was assessed by filtering out unhealthy and dying cells, after considering the number of genes detected, their counts and the proportion of mitochondrial genes. The cells with transcripts number below 500 or exceeding 8000 and transcript counts exceeding 30000 as well as those with a mitochondrial gene percentage more than 10% per cell, were excluded from further analysis (FIG. 10D).

### Normalization, Highly Variable Gene Selection, Scaling, and Dimensionality Reduction

After filtering out unhealthy cells, the dataset was normalized by log normalization. The log-transformed values are then centered across all genes for each cell, resulting in the relative expression levels across different cells. Next, 3000 highly variable genes were selected, which are highly expressed in some sets of cells and at very low levels in other sets of cells. These genes have significantly higher variance across cells than by chance. The genes with high variance are likely to be more informative and may represent the biologically meaningful differences between cell types or states. After highly variable genes are selected, to negate the effect of their high expression difference, data is scaled to bring the expression levels of genes within the range. Scaling helps to remove the unnecessary source of variation of the data by bringing the distribution of values between 0 and 1. To reduce the dimensionality of the large matrix to small matrix with less factors, principal component analysis (PCA) was performed using the first 50 PCAs and highly variable genes selected (which were scaled in the previous step) were used for the PCA input. The PCA projects a high dimensional dataset to very few dimensions. The top few principal components capture the majority of variance of the data are kept for the downstream steps.

Batch Correction, Cell Clustering and cell Type Identification

The PCA transformed data is fed into the batch correction step by harmony method using R package harmony_1.2.0. It is fast and removes technical variations introduced by batch effects, while preserving biological variability across batches and experimental conditions. Next, to visualize the highly complex data, UMAP was generated UMAP using harmony embeddings. Cell clustering and cell type identification was done with the batch corrected data. Seurat uses graph-based clustering method. The shared nearest neighbor (SNN) identifies cell to cell relationships based on the nearest neighbor graph to define cell neighborhoods. To cluster the cells, Louvain algorithm was used iteratively to group cells together. It tries to determine the modularity of the graph. In network analysis modularity is a commonly used concept to identify the community structure within a graph. It measures how well communities can be partitioned into distinct structures and suggest a strong community. A resolution parameter of 0.6 was used while clustering and resulted in 28 clusters (0-27) overall. Canonical cell type markers were used to identify the distinct brain cell types (Table 3, FIG. 10E).

TABLE 3
Canonical Marker Genes for Identifying
UMAP Clusters and Annotated Cell Types

| Marker | Cell | Marker | Cell | Marker | Cell |
|---|---|---|---|---|---|
| Genes | Type | Genes | Type | Genes | Type |
| Tmem119 | MG | Pdgfra | OPC | Cldn5 | Endo |
| Cx3cr1 | MG | Cspg4 | OPC | Cdh5 | Endo |
| Ptprc | MG | St8sia3 | OPC | Slc2a1 | Endo |
| Fcrls | MG | Olig2 | OPC | Abcb1a | Endo |
| P2ry12 | MG | Sox10 | OPC | Vwf | Endo |
| Aif1 | MG | St8sia1 | OPC | Mfsd2a | Endo |
| Hexb | MG | Cd9 | OPC | Lef1 | Endo |
| Slc2a5 | MG | Neurod1 | Neurons | Sox17 | Endo |
| Siglech | MG | Bsn | Neurons | Cd19 | Immune |
| Trem2 | MG | Dcx | Neurons | Cd79a | Immune |
| Itgam | MG | Gap43 | Neurons | Actb | Immune |
| Gfap | Astro | Ncam1 | Neurons | Cd3d | Immune |
| Aqp4 | Astro | Syp | Neurons | Cd3e | Immune |
| Gldc | Astro | Rbfox3 | Neurons | Cd3g | Immune |
| Slc1a3 | Astro | Nes | Neurons | Ptprc | Immune |
| Slc1a2 | Astro | Tubb3 | Neurons | Ms4a1 | Immune |
| Aldh1l1 | Astro | Tbr1 | Neurons | Ccr7 | Immune |
| Dab1 | Astro | Stmn1 | Neurons | Ms4a7 | Immune |
| Fam107a | Astro | Slc5a7 | Neurons | Col1a2 | VLMC |
| Agt | Astro | Chat | Neurons | Col1a1 | VLMC |
| Mbp | Oligoden- | Slc17a8 | Neurons | Mgp | VLMC |
| | drocytes | | | | |
| Sox10 | Oligoden- | Slc17a6 | Neurons | Lum | VLMC |
| | drocytes | | | | |
| Mog | Oligoden- | Gad1 | Neurons | Dcn | VLMC |
| | drocytes | | | | |
| Car2 | Oligoden- | Gad2 | Neurons | Pdgfra | VLMC |
| | drocytes | | | | |
| Cnp | Oligoden- | Slc6a5 | Neurons | Serpinf1 | VLMC |
| | drocytes | | | | |
| Plp1 | Oligoden- | Syn1 | Neurons | Aqp1 | Choroid |
| | drocytes | | | | plexus |
| Opalin | Oligoden- | Syn2 | Neurons | Folr1 | Choroid |
| | drocytes | | | | plexus |
| Mobp | Oligoden- | Syn3 | Neurons | Klk1b5 | Choroid |
| | drocytes | | | | plexus |
| Olig1 | Oligoden- | Pdgfrb | Pericytes | Lama1 | Choroid |
| | drocytes | | | | plexus |
| Olig2 | Oligoden- | Rgs5 | Pericytes | S100b | Choroid |
| | drocytes | | | | plexus |
| Cldn11 | Oligoden- | Cspg4 | Pericytes | Otx2 | Choroid |
| | drocytes | | | | plexus |
| Mag | Oligoden- | Vtn | Pericytes | Slc4a5 | Choroid |
| | drocytes | | | | plexus |
| Galc | Oligoden- | Higd1b | Pericytes | Igf2 | Choroid |
| | drocytes | | | | plexus |
| Flt3 | Dendritic | S1pr3 | Pericytes | | |
| | cells | | | | |
| Zbtb46 | Dendritic | Mcam | Pericytes | | |
| | cells | | | | |
| | | Ifitm1 | Pericytes | | |
| | | Ehd3 | Pericytes | | |
| | | Atp13a5 | Pericytes | | |

### Differential Gene Expression and Heatmaps of Microglia Subset

The differential gene expression analysis was performed on microglial subset using R package DESeq2_1.44.0. Since the count dispersion of features across samples is quite high, and to account for this overdispersion in scRNAseq data, DESeq2 uses negative binomial model to obtain a better statistical significance measure. DESeq2 uses the Wald test for pair wise analysis between groups. The raw p-values were calculated using the Wald test which is a test of hypothesis usually performed on parameters that have been estimated by maximum likelihood. The significant genes were filtered both by raw p-values (<0.05) and adjusted p-value (padj).

### Trajectory Analysis

The preprocessed, integrated, and batch corrected microglia single cell RNA sequencing data (microglia Seurat object) was used as the input for trajectory analysis. Trajectory analysis was performed using the monocle3_1.3.7 package in R to understand the developmental trajectory of single cells. Initially, the Seurat object is converted to cell data set object (cds) and the cds object was further pre-processed by following steps. The cds were preprocessed using the preprocess_cds( ) function, which normalized the data using principal component analysis (PCA) with the first 50 dimensions. UMAP was used to reduce dimensionality to two axes. A trajectory graph was constructed by learn_graph( ) function. Once the trajectory graph was constructed, the cells were ordered along the trajectory depending on their progress through the development program. The one-month untreated wild type group (WT.1m.Un) was selected as the root node in the current analysis since this time point was only a week after the pups were released from weaning. The trajectory graph was visualized using UMAP plot. The pseudotime values generated for all the 12216 cells and stored in the cds. The pseudotime vector stored in the cds was extracted and added to the metadata of microglia Seurat object. The continuous pseudotime vector was further converted to 25 categories (25 bins) and the resulting vector was stored as a new variable “pseudotime_bin” in the metadata of microglia Seurat object. Next, the proportion of each group in the bins was calculated and plotted as a trajectory vs. proportion graph.

### Module Score Analysis

To assess the activity of specific gene sets in individual cells, module score analysis was performed using AddModuleScore function of the Seurat_5.1.0 package in R. Module scores were calculated for Homeostatic, Stage 1 DAM and Stage 2 DAM gene sets. After splitting the microglia Seurat object based on group variable (there are 10 groups), the module score was calculated for each group from individual cells. It calculates a score for each cell representing how strongly a set of modules (set of genes), are expressed within a cell, compared to a set of randomly selected background control genes. The control genes were chosen with similar expression levels to ensure that the module score is not confounded by overall gene expression differences between cells. As data may not follow a normal distribution, the Wilcoxon rank sum test is used as default test to compare module genes to control genes.

### Novel Object Recognition Assay

The object recognition test was used to assess memory in mice. The assay was conducted on three consecutive days. On day 1, mice were individually placed into a plastic container with black walls of size 37×29×17 cm³ and allowed to explore the arena for 10 minutes. On day 2, two identical objects were placed in the center (kept at equidistance from the wall and each other) and the time interacting with each object was recorded over 10 minutes. On the third day, one object was removed and replaced by a novel object. Lego towers and T25 tissue culture flask filled with white gravel were used as objects. Mice were placed in the arena for ten minutes and timed for interaction with each object. The arena and objects were cleaned with 70% ethanol between each mouse to eliminate olfactory cues. The discrimination index was calculated by using a formula, TN-TF/TN+TF, where TN and TF are time spent exploring novel and familiar objects, respectively.

### Von Frey Assay

Tactile sensitivity is measured by studying paw withdrawal frequency (PWF) using a series of von Frey filaments with incremental filament thickness. The mice are placed into an acrylic box and habituated in a quiet room for an hour. Mechanical stimulus applied perpendicularly to the mid-plantar surface of hind paws for 2 seconds with enough force to bend the filament. Each paw was stimulated 5 times by each filament with an interval of 5 seconds between each measurement. Only rapid paw withdrawal or prolonged paw withdrawal combined with flinching or licking of the paw were recorded. 50% paw withdrawal threshold was calculated. The left and right hind paws of each mouse were assessed on three consecutive days and the average withdrawal threshold of each paw was used to plot the data. A lower 50% threshold value suggests higher tactile sensitivity.

### Immunofluorescence Assay and Microscopy

The brain hemisphere was immersion fixed in 10% neutral buffered formalin (~4% paraformaldehyde) for 20-22 h and paraffin-embedded. 5 μm thick sagittal sections spaced at 40 μm collected at 4-6 different depths were processed for DCX staining. After dewaxing and hydration, DCX antigen was retrieved by boiling the sections in acidic condition for 30 min followed by a 20-minute incubation at room temperature as the sections cool off. After blocking in 2% goat serum, sections were incubated with anti-DCX antibody (Santa Cruz Biotechnology, sc-271390, 1:50) overnight at 4 °C. FITC conjugated secondary IgG (MP Biomedicals) was used at 1:1000 dilution. Sections were mounted using Vectashield (Vector Laboratories) containing DAPI for staining nuclei and visualized by fluorescence microscopy. DCX labeled brain sections were visualized by Olympus IX inverted fluorescence microscope using 40× oil-immersion objective lens (NA1.35) and the number of DCX+ cells in the inner margin of entire SGZ was manually counted. Digital images of consecutive 20 optical sections of 0.2 μm thickness were collected by Photometrix cooled CCD camera (CH350/LCCD) driven by DeltaVision software from Applied Precision (Seattle, WA, USA). DeltaVision software (softWoRx) was used to deconvolve these images. The images presented are maximum intensity projections of all 20 optical sections. Images were analyzed using softWoRx or ImageJ software (NIH, MD, USA).

### Immunohistochemistry, Image Acquisition, and Analysis

Tissue embedding and immunohistochemical staining of sagittal brain sections was performed by NeuroScience Associates (Knoxville, TN, USA). Thirty-micron thick sections were cut through the entire depth of the brain. Free-floating sections after blocking were incubated in rabbit anti IBA-1 antibody (Abcam, ab178846, 1:75000) or anti GFAP antibody (Dako, Z0334, 1:14000) for overnight at room temperature. Biotinylated secondary antibodies were from Vector Laboratories (BA-1000, 1:1000). Sections were developed following standard methods. To quantify microglial abundance in hippocampus, IBA1 stained brain slices from 6-7 depths at the interval of 180 μm were analyzed and bright field images were captured using A10 PL 10× (for counting) objective lens (Nikon) with a numeric capture of 0.25. After applying constant threshold numbers were automatically counted using ImageJ. For analyzing astrocytes, 4-5 random images of GFAP-stained hippocampus at 3 depths spaced at 180 μm from each mouse were captured using 40× oil-immersion objective lens (Plan-Apochromat, Nikon) with a numeric aperture of 1.3. Both objective lenses (10× and 40×) were connected to the Nikon Olympus microscope supported by Nikon digital DS-Fi1-U2 camera controlled by NIS-Elements F3.0 Nikon software. GFAP positive areas were highlighted by applying constant threshold and percentage-stained areas were quantified using ImageJ.

### Morphometric Assessments of Microglia Using Imaris

For analyzing microglia extensions and soma, 2 random images of hippocampus (stained for IBA1 as described above) at 2 depths (4 images/mouse) spaced at 180 μm were acquired using 40× oil-immersion objective lens. For morphometric measurements of microglia extensions and soma, images were processed using the Imaris 9.5.1 algorithm (Bitplane, UK). All measurements were done on 2-dimensional images after converting bright filed images to ‘ims’ files. Structural complexity of extensions was analyzed by quantifying cumulative length of filaments, number of branches and terminal points. Extensions originating from soma were identified and automated tracing was performed by ‘auto path’ feature in filament wizard. The origin of filaments from the soma of single microglia was individually verified. Microglia present at the edges or soma touching each other were excluded from the analysis. Microglia with intermingled extensions or filament with incorrect connections (seen in less than 5%) were manually removed. For extensions, 400-500 hippocampal microglia (40±10 per mouse) per experimental group were reconstructed whereas cell soma was identified via surface creation wizard in 600-700 (60±15 per mouse) microglia. For soma, a constant threshold was set to exclude branches and extensions.

### Statistical Tests

Graphs were plotted in MS Excel or GraphPad Prism 8.4.3. For comparing two groups student's t-test or Mann-Whitney U test was performed. For comparing more than two groups, unless specified, one-way ANOVA with Tukey's posthoc analysis (parametric) or Kruskal-Wallis test (nonparametric) was performed. All statistical tests were done with GraphPad Prism 8.4.3 and were two-tailed. Statistical test and P values are indicated in the figure legends. P<0.05 was considered significant. *P<0.05, **P<0.01, ***P<0.001, ****P<0.0001.

### Results

### Reduction of the Open Chromatin Mark H3K4Me3, Hippocampal Post-Natal Neurogenesis and Microglia in The Brain of Kmt2d^{+/βGeo} (Kbk) Mice

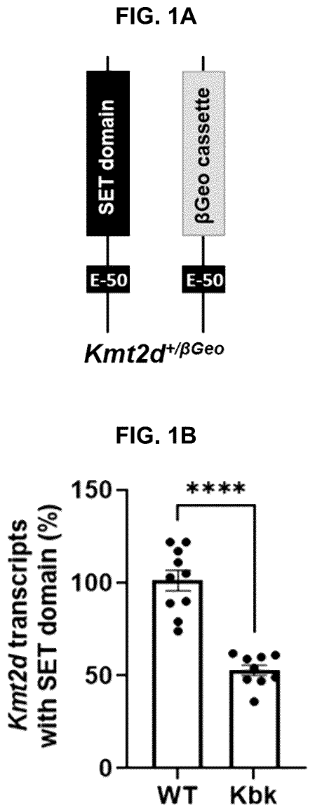

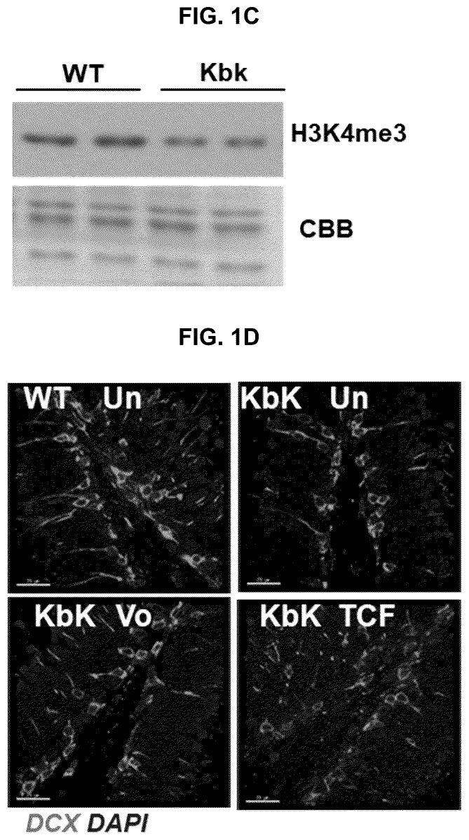

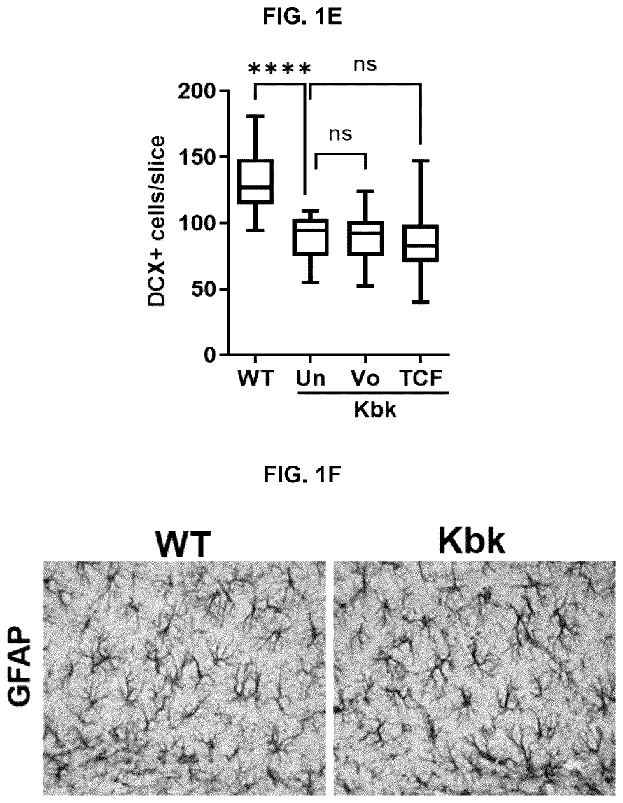

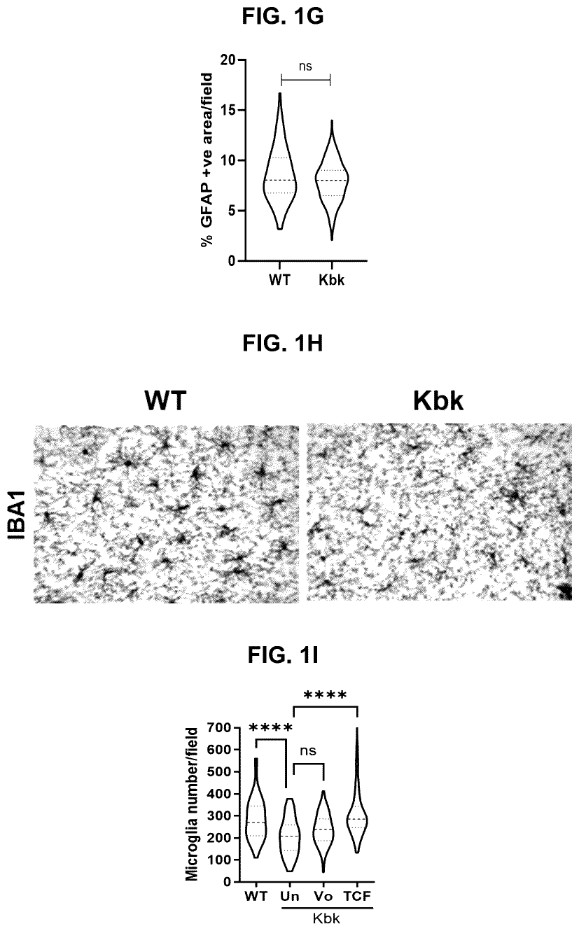

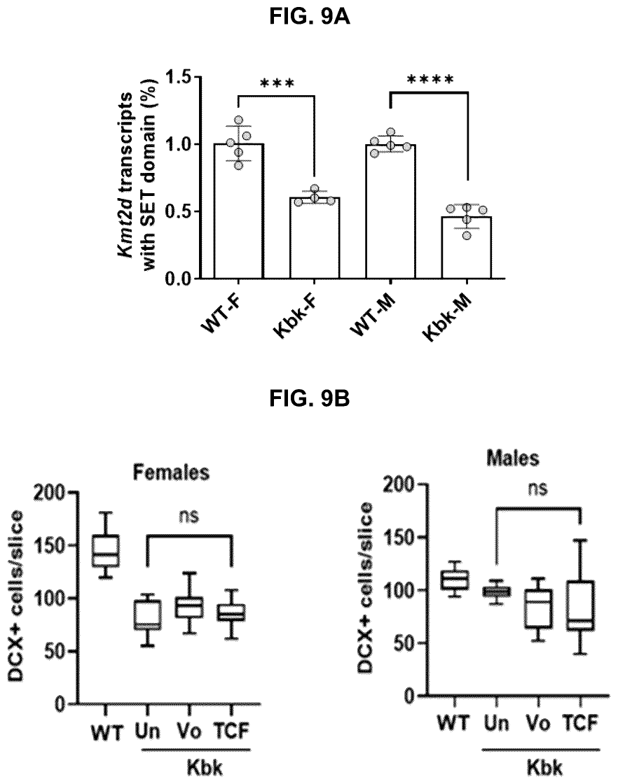

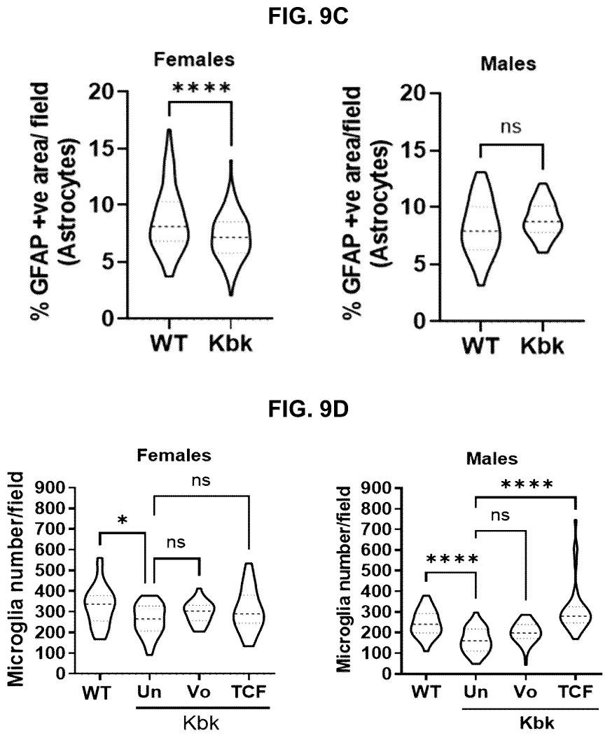

In the Kmt2d deficient mouse the catalytic SET (suvar, enhancer of zeste, trithorax) domain in one allele is replaced by a B Geo cassette (Kmt2d^{+/βGeo}; FIG. 1A). Mice backcrossed to 99-100% to C57BL/6J background were confirmed for loss of the SET domain in ~50% of Kmt2d transcripts in the brain (FIG. 1B, FIG. 9A) and reduction in tri-methylation of histone 3 (H3K4me3) (FIG. 1C). Bjornsson et al reported that Kbk mice showed decrease in post-natal neurogenesis (as measured by staining for doublecortin X (DCX, a marker for neural precursors and immature neurons), in the subgranular zone (SGZ) of the dentate gyrus (DG) of the hippocampus, that could be increased by administration of a pan HDACi AR-42. As shown in FIG. 1D-E and FIG. 9B, the present study also found that Kbk mice show a reduction in staining of DCX compared to WT counterparts. The TCF (DMSO+PEG+HP-β-CD+Vorinostat (Vo)) was administered in once weekly injections: this regimen was known to be brain permeant and well tolerated in long term administration in mice. However, even after three months, neither the TCF nor Vo (the active HDACi ingredient in TCF) increased DCX or stimulated post-natal hippocampal neurogenesis in Kbk mice (FIG. 1D-E and FIG. 9B).

The study next focused on the brain's major immune cells, astrocytes and microglia, whose characteristics remain unknown in KS mice. As shown in FIG. 1F-G, astrocytes stained for their characteristic marker Glial Fibrillary Acid Protein (GFAP) showed no significant change in Kbk compared to WT mice (FIG. 1F-G; minor changes seen with individual genders in FIG. 9C, did not prevail when both were combined). Microglia were subsequently examined, which are known to be present in higher density in the hippocampus. As shown in FIG. 1H-I and FIG. 9D, microglia were stained with their characteristic marker, IBA1 (Ionized calcium-binding adaptor protein-1 an actin-binding protein). Notably, in Kbk mice, the maximal and median microglial densities were lowered by 33% and 23% compared to wild type counterparts. TCF (but not its independent components) quantitatively increased microglia densities in the hippocampus (FIG. 1H-I, FIG. 9D). Together these data suggested that hippocampal microglia are reduced in Kbk mice. Moreover, although the TCF failed to stimulate post-natal neurogenesis in the SGZ of the DG, it may influence microglial function in the hippocampus.

Single Cell RNA Sequencing of Wild Type and Mutant Microglia and their Responses to Chronic TCF-Administration

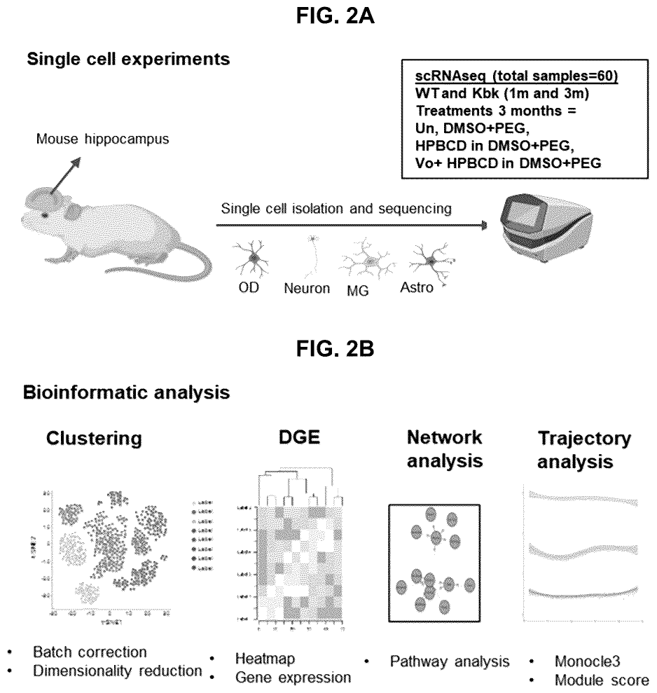

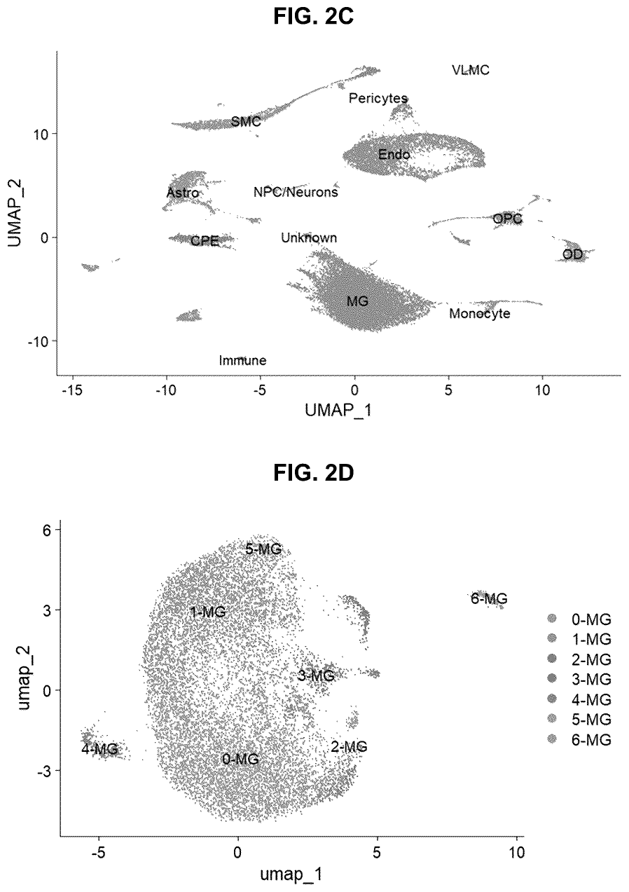

To understand molecular changes in hippocampal microglia elicited by TCF, single cell RNA sequencing of cells isolated from WT and Kbk mice was compared in the presence or absence of treatment. As summarized in FIG. 2A and FIG. 10A, a total of 60 mice were utilized (balanced for sex and genotype). This included a cohort of six mutant and six WT mice at one month of age that provided a baseline to compare early post-natal differences, as well as treatment groups of 24 WT and 24 Kbk at three months of age, that received weekly TCF or control treatments of PEG (in DMSO) and/or HP-β-CD (in DMSO+PEG). Single-cell preparations of isolated hippocampi were hash tagged (to distinguish between WT and Kbk; FIG. 10B-D) before advancing to single call capture, library preparation, RNA seq cell ranger and Seurat Analysis. After filtering, a total of 25, 165 high-quality healthy single cells were obtained. These included 12,216 microglia (Table 4), confirming their enrichment in the preparations. Uniform Manifold Approximation and Projection (UMAP) analysis was used to reduce the multidimensional scRNA-seq data into 28 distinct clusters that were categorized into 11 major cell types which included of microglia, endothelial cells, astrocytes, choroid plexus epithelial cells, oligodendrocytes, oligodendrocyte progenitor cells, dendritic cells, pericytes, neural progenitor cells/neurons, vascular leptomeningeal cells, and immune cells (Table 4 and FIG. 2C). Microglia with 12,216 cells comprised 48.5% of total and were judged to be suitably abundant for further single cell analyses (while the remainder groups were not; Tables 3-6 (DCF-double combination formulation). The microglial subsets were pre-processed before performing UMAP analysis, which revealed presence of seven clusters delineated computationally (0-MG to 6-MG) (FIG. 2D) suggesting they may span multiple states, across genotypes and treatments.

TABLE 4
Quantified Cell Types Identified from the UMAP Clusters

| | Cell | Percentage |
|---|---|---|
| | Number | (%) |
| Total | 25165 | 100 |
| Cell Types | | |
| Microglia (MG) | 12216 | 48.5 |
| Endothelial cells (Endo) | 5148 | 20.5 |
| Pericytes | 2224 | 8.8 |
| Astrocytes (Astro) | 1682 | 6.7 |
| Choroid plexus epithelial cells (CPE | 1047 | 4.2 |
| Oligodendrocyte progenitor cells (OPC) | 716 | 2.8 |
| Oligodendrocytes (OD) | 860 | 3.4 |
| Immune cells | 845 | 3.4 |
| Dendritic cells (DC) | 237 | 0.9 |
| Vascular leptomeningeal cells (VLMC) | 116 | 0.5 |
| Neural progenitor cells/neurons (NPC/Neurons) | 74 | 0.3 |

TABLE 5
Cell Type Number per Group

| | | | WT 3 | | | | | Kbk | | |
|---|---|---|---|---|---|---|---|---|---|---|
| | | | m | | | Kbk | Kbk | 3 m | Kbk | Kbk |
| | WT 1 | WT 3 | DMSO | WT 3 | WT 3 | 1 m | 3 m | DMSO | 3 m | 3 m |
| | m Un | m Un | PEG | m DCF | m TCF | Un | Un | PEG | DCF | TCF |
| MG | 1490 | 1040 | 1144 | 1219 | 1890 | 1003 | 982 | 1191 | 907 | 1350 |
| Endo | 602 | 437 | 378 | 487 | 780 | 411 | 456 | 480 | 461 | 656 |
| Pericytes | 242 | 169 | 150 | 164 | 303 | 247 | 232 | 209 | 239 | 269 |
| Astro | 330 | 167 | 91 | 66 | 123 | 145 | 155 | 123 | 188 | 294 |
| CP | 97 | 114 | 43 | 32 | 156 | 138 | 71 | 75 | 122 | 199 |
| OPC | 98 | 61 | 38 | 27 | 79 | 47 | 44 | 55 | 83 | 184 |
| OD | 116 | 89 | 49 | 33 | 82 | 64 | 96 | 79 | 80 | 172 |
| Immune Cells | 83 | 76 | 81 | 76 | 108 | 77 | 86 | 97 | 56 | 105 |
| DC | 37 | 13 | 19 | 28 | 19 | 26 | 15 | 37 | 19 | 24 |
| VLMC | 11 | 11 | 5 | 6 | 8 | 14 | 12 | 15 | 6 | 28 |
| NPC/Neuron | 9 | 6 | 9 | 4 | 11 | 8 | 5 | 8 | 6 | 8 |

TABLE 6
Microglia Number per Group

| | | MG |
|---|---|---|
| | Group | Number |
| 1 | Kbk 1 m Un | 1003 |
| 2 | Kbk 3 m DCF | 907 |
| 3 | Kbk 3 m DMSO_PEG | 1191 |
| 4 | Kbk 3 m TCF | 1350 |
| 5 | Kbk 3 m Un | 982 |
| 6 | WT 1 m Un | 1490 |
| 7 | WT 3 m DCF | 1219 |
| 8 | WT 3 m DMSO_PEG | 1144 |
| 9 | WT 3 m TCF | 1890 |
| 10 | WT 3 m Un | 1040 |

### Downregulation of Hippocampal Microglia Seen in Kbk Mice is Mitigated by TCF.

The study initiated differential gene expression (DGE) analysis to investigate changes in transcriptional signatures of microglia across experimental groups. Due to the sparsity and high noise inherent in scRNA-seq data, traditional DGE approaches were not used, but rather a pseudobulk analysis, which aggregates single-cell expression counts from individual samples, effectively transforming the data into a bulk RNA-seq format.

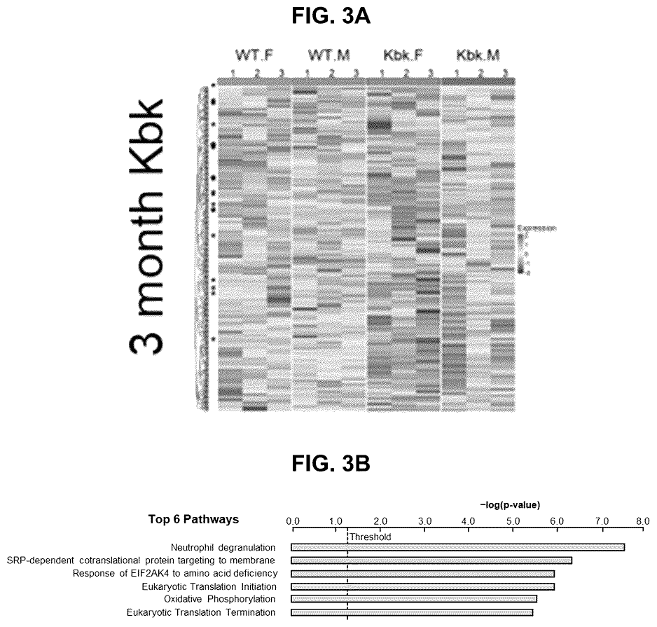

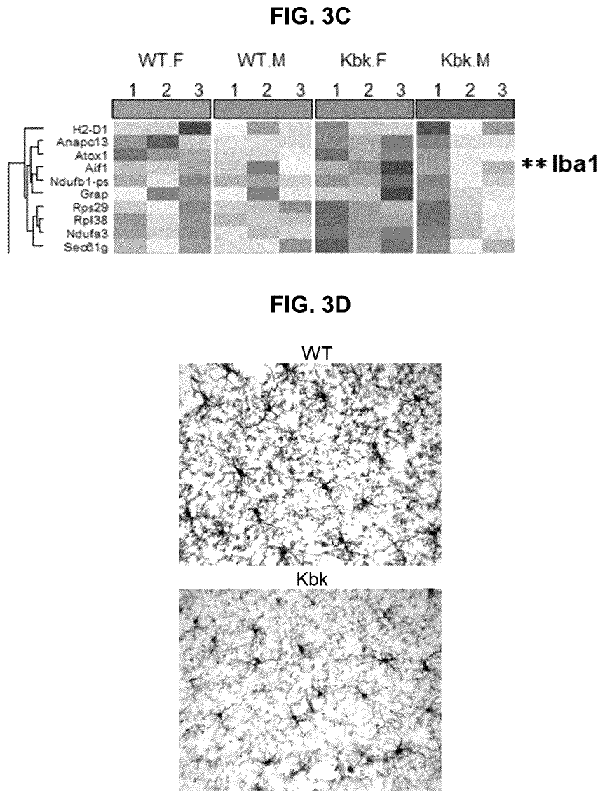

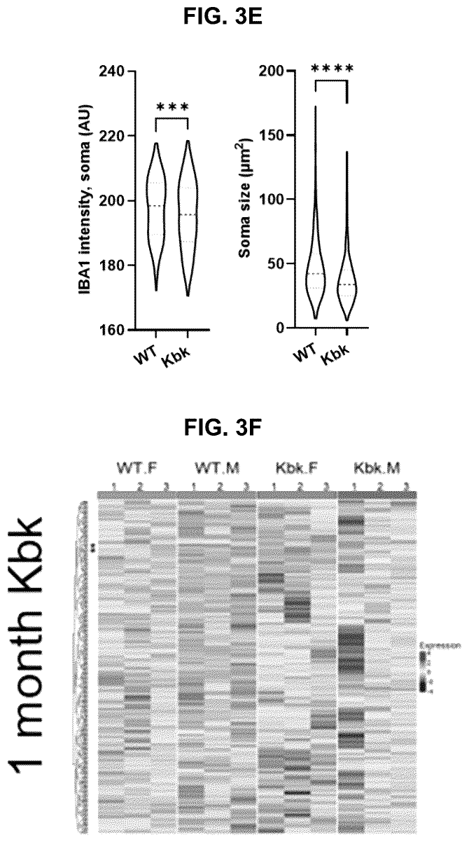

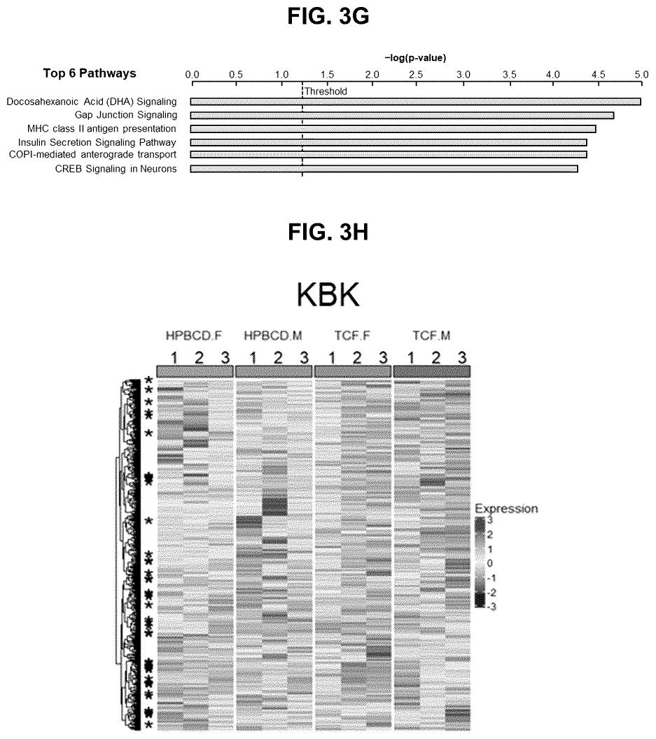

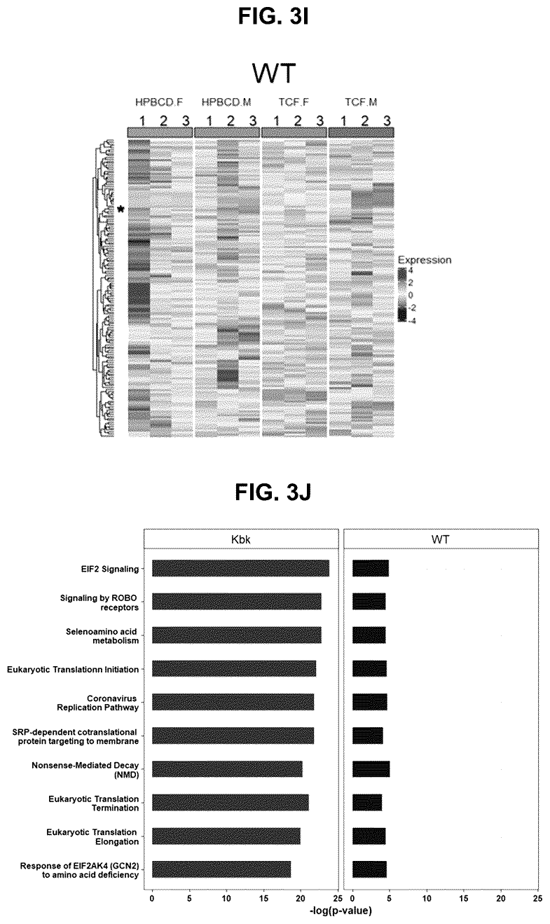

Comparisons of DGEs of untreated WT and Kbk mice at three months are shown in FIG. 3A. Here, three female and one male Kbk sample formed a distinct cluster, while the remaining two males grouped more with WT counterparts. This type of heterogeneity is characteristic of defect in Kmt2d, since the extent of loss of chromatin and consequences for gene expression, can vary between mutant animals. The major cellular pathways impacted were investigated using Ingenuity Pathway Analyses (IPA; FIG. 3B; FIG. 11B-C). Of the top six predicted pathways, five suggested global reduction in translation and oxidative phosphorylation (FIG. 3B). Notably, Aif1 (the gene coding for the major microglial marker IBA1) was in the top ten downregulated transcripts identified (p<0.05, fdr <0.05; FIG. 3C). IBA1 was also confirmed to be reduced in hippocampal Kbk microglia detected by immunohistochemical (IHC) which were additionally reduced in soma size (FIG. 3D-E). Thus, at 3 months, Kbk microglia appear to be arrested forms, small and downregulated in basic cellular pathways and suppressed in a major proliferative, inflammatory maker IBA1. Examination of one month old mice also showed heterogeneity in microglia, but at this age the major changes in mutants were in signaling and vesicular pathways (FIG. 3F-G). Together, the data in FIG. 3A-G suggest that changes in hippocampal mutant microglia emerged as early as one month after birth but global reduction in cellular translation, energy production and IBA1 and progression to small, arrested forms. This is in contrast to findings that in normal mice early microglia display a round amoeboid morphology, active proliferation and phagocytosis

To examine the effects of TCF, mice were first administered at one month of age and followed up weekly for up to 3 months. As shown in FIG. 3H-I, by DEG analyses, TCF treatment improved clustering in five out of six Kbk mice relative to control HP-β-CD-treated samples. A clustering pattern emerging between DMSO+PEG and HP-β-CD (in DMSO+PEG) treatments was also observed (FIG. 11A). But these were distinct from TCF-induced clustering seen in treated mutants. IPA analyses (FIG. 3J) suggested that in Kbk, TCF treatment globally stimulated translation and other cellular pathways, whereas in WT mice, TCF mildly suppressed a small subset of genes and pathways (3H-J). Together these data suggest that in Kbk, TCF effectively reversed massive transcriptional down-regulation associated with three-month old mutants but WT with normal heterochromatin were protected from TCF.

### Chronic TCF Administration Selectively Stimulates Microglial Histone H3 and Overall Increase in H3K4 Open Chromatin Mark in Kbk Brain

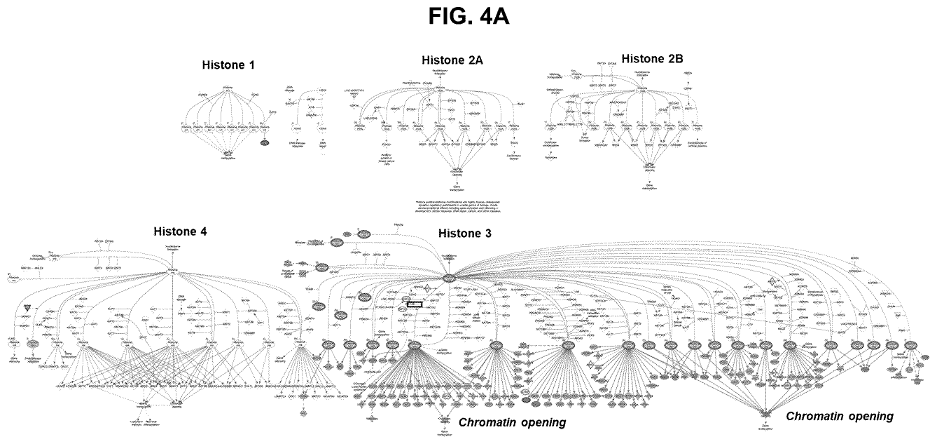

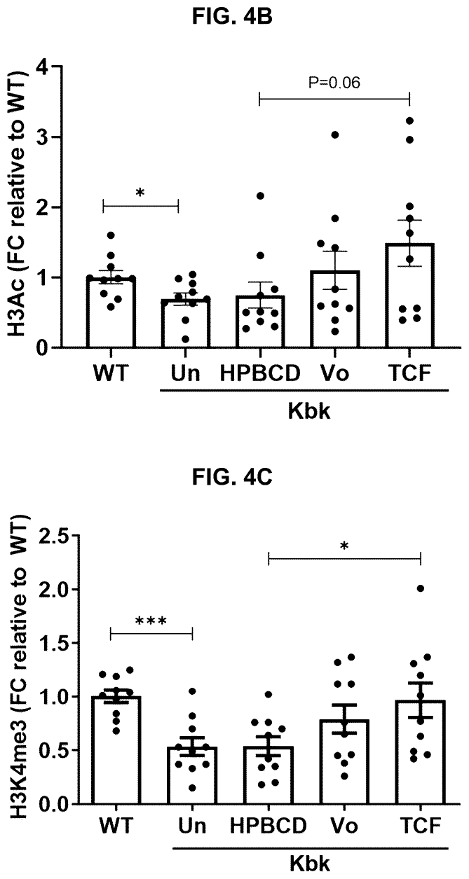

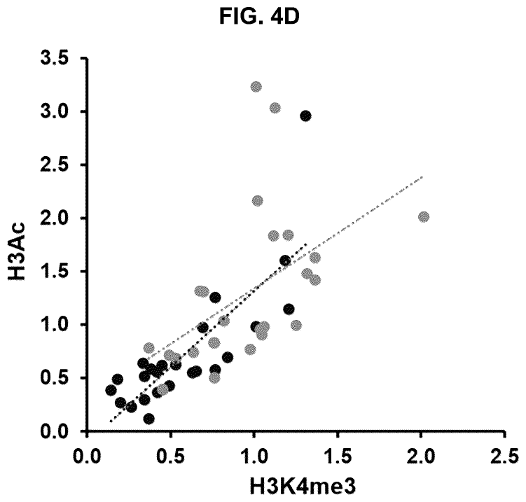

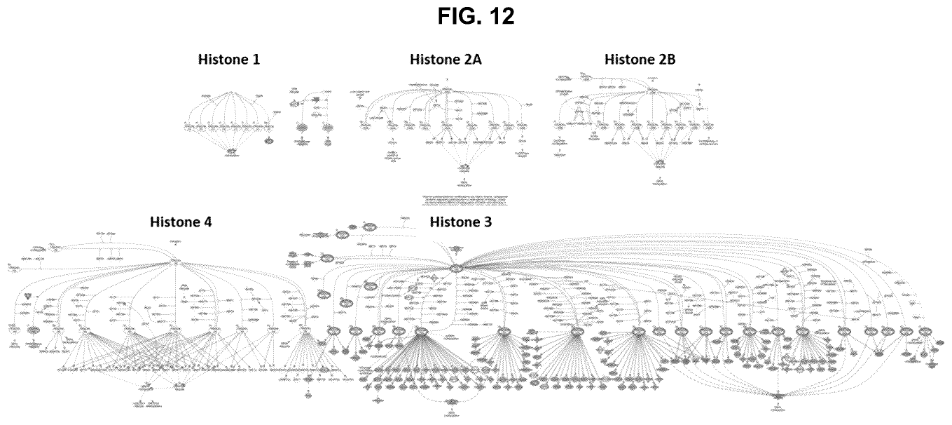

Histone proteins in the nucleus are known to play a key role in the maintenance of chromatin. Vorinostat (Vo), the HDACi in TCF blocks deacetylation of histones by targeting multiple HDACs. IPA analyses predicted elevation of the chromatin mark H3K4me3 in Kbk, without stimulation of Kmt2d but unexpectedly driven by increase of a single histone H3 (FIG. 4A; FIG. 12). Importantly, the other histones (1, 2 and 4) and associated pathways were effectively unperturbed, suggesting chronic TCF resulted in selective, long-term targeting through histone H3.

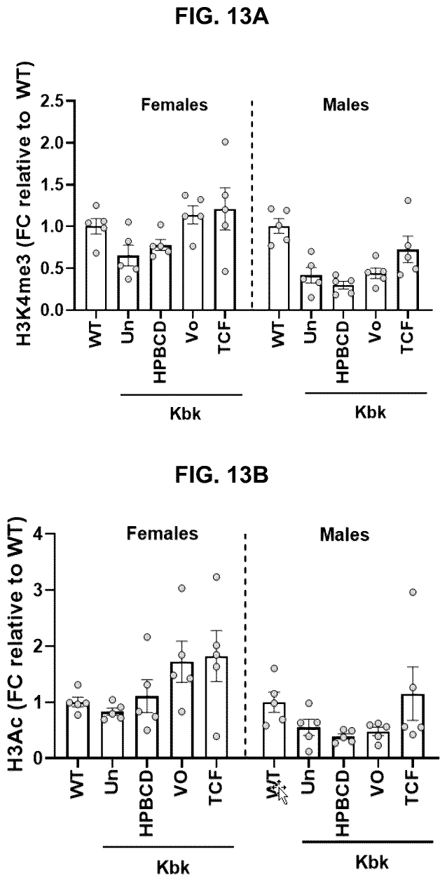

As further validation, the study examined the utility of TCF to stimulate histone H3 acetylation and the open chromatin mark in the whole brain. As shown in FIG. 4B, after 3 months of weekly injections, TCF appeared to increase median values of H3Ac levels in the Kbk brain compared to HP-β-CD (in PEG+DMSO; that lack HDACi (Vo; but with a p-value of 0.06). Vo (in DMSO+PEG) may also have an effect but based on H3Ac levels alone, a firm conclusion on the significance of TCF-induced acetylation could not be made. No sex specific effects were seen (FIG. 13A-B). However, as shown in FIG. 4B, TCF induced a significant increase of the open chromatin mark H3K4me3, relative to HP-β-CD (in PEG+DMSO). Vo (in PEG+DMSO) also increased levels of H3K4me3 but with intermediate values that could not be separated as statistically significant from TCF or HP-β-CD (in PEG+DMSO). Notably (and as shown in FIG. 4C), in >90% of animals there was a strong positive correlation between acetylation and trimethylation which was seen in both genders (FIG. 4D). Only three of the fifty mice showed disproportionately elevated levels of acetylation. Together, these data established a primary effect of histone H3 mediated acetylation-dependent promotion of H3K4me3 in the Kbk brain. TCF achieved higher levels of acetylation and trimethylation than its components, suggesting its brain permeant and HDACi-dependent properties were important for acetylation-dependent histone trimethylation throughout the brain.

### TCF Treatment Drives Divergent Microglial Trajectories in WT and Kbk Mice

The distinct morphological differences observed in microglia from Kbk and WT mice (in FIG. 3A-J) led us to hypothesize that these variations reflect underlying molecular differences in cell states. To investigate this, the scRNA-seq dataset was revisited and pseudotime analysis was applied, a computational technique that organizes cells along a theoretical trajectory, representing their progression through biological processes. Pseudotime analysis enables the reconstruction of temporal events at a single-cell resolution, providing a deeper understanding of complex cellular dynamics. Using the R package Monocle3, a microglia trajectory was constructed from 12,216 cells across all experimental groups, assigning pseudotime values to each cell. Cells at the beginning of the trajectory represent earlier, less differentiated states, while those at later stages represent more advanced differentiated states. To quantify these trends, pseudotime values were divided into 25 bins, and the proportion of cells from each experimental group was calculated within these bins. WT untreated-mice at 1 month of age were taken to be the start of the trajectory.

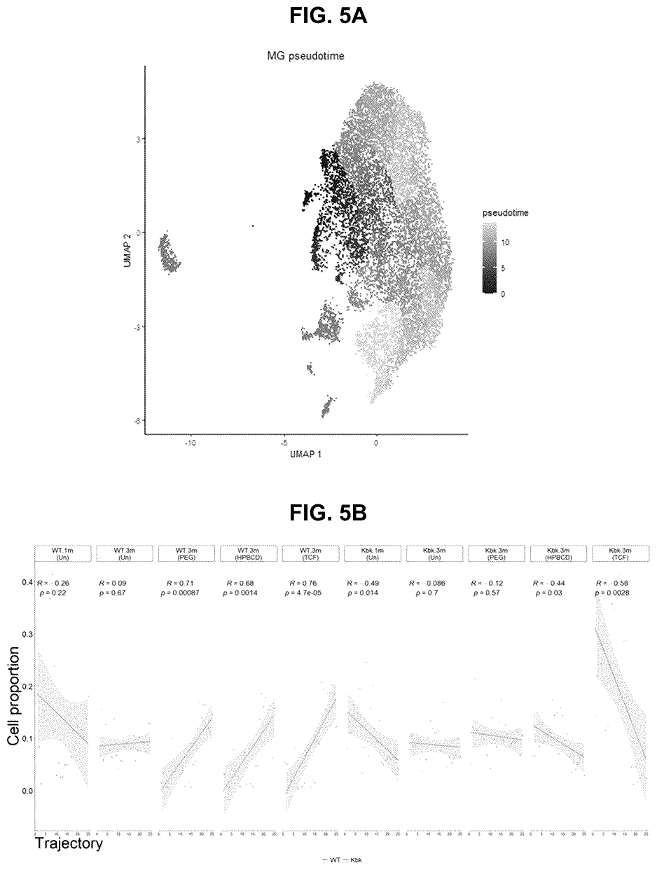

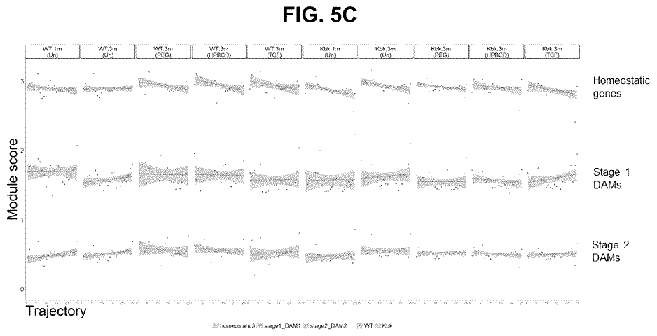

As shown in FIG. 5A-B, microglial trajectories exhibited distinct patterns between Kbk and WT groups under different treatment conditions. For Kbk mice, the proportion of cells treated with PEG (+DMSO), HP-β-CD (in DMSO+PEG), and TCF decreased along the pseudotime trajectory, with Pearson correlation R=−0.12, p=0.57; R=−0.44, p=0.03; R=−0.58, p=0.0028, respectively). In contrast, WT mice treated with PEG (+DMSO), HP-β-CD (in DMSO+PEG), and TCF showed increased proportions along the pseudotime trajectory (Pearson correlation R=0.71, p=0.00087; R=0.68, p=0.0014; R=0.76, p=4.7×10⁻⁵, respectively). These opposing trends highlight a fundamental divergence in how WT and Kbk microglia progress through their cellular states.

While different treatments did not significantly alter the linear relationship between pseudotime and cell proportions in WT microglia, TCF exerted a marked effect on Kbk microglia (FIG. 5B). Specifically, TCF led to a significant decrease in cell proportions along the pseudotime trajectory compared to HP-β-CD treatment. This effect suggests that TCF influences the progression of Kbk microglia to drive their differentiation toward earlier, distinct cellular states and outcomes (driving toward the start of the trajectory set at WT at one month). In contrast, WT microglia initially appeared in lower proportions at early pseudotime stages, but their numbers increased as pseudotime progressed, indicating a gradual advancement (without differentiation) toward later states.

Module Score vs. Pseudotime Analysis Suggested that the Distinct Molecular Gene Modules Responsible for Microglial Activation were Unchanged by TCF or its Components In a wide range of neurodegenerative disorders such as Alzheimer's disease, amyotrophic lateral sclerosis, and multiple sclerosis, the expression of both stage 1 and stage 2 disease associated microglia (DAM) genes, as well as homeostatic genes, is differentially regulated. DAM arise from homeostatic microglia, which normally play a crucial role in maintaining the health of the central nervous system (CNS) by clearing debris and supporting neural circuits. The shift from homeostatic microglia to an activated DAM state is a key process in neuroinflammation by upregulation of genes associated with inflammation, lipid metabolism, and phagocytosis (such as Lpl, Cst7, and Axl). The DAM is associated with increased expression of DAM gene modules (Stage 1 DAM genes:—Tyrobp, Ctsb, Apoe, B2m, Fth1, Trem2, and Ctsd, Stage 2 DAM genes:—Lpl, Cst7, Axl, Itgax, Spp1, Cd9, Ccl6, Csf1, Trem2, Timp2 and Ctsl) and decreased expression of homeostatic genes (Hexb, Tmem119, Cx3cr1, P2ry12, P2ry13, Cst3, Cd33, Csf1r, Ctss, Tmsb4×, and C1qb). The DAM cells are activated by Trem2 signaling pathway and this activation leads to the transition from homeostasis to neuroinflammation. FIG. 5C illustrates the module score trends along the pseudotime axis across the different experimental treatments in Kbk and WT mice. From the module score vs. pseudotime analysis, there was no major difference in homeostatic, stage 1, and stage 2 DAM gene modules across any treatments, therefore, implying that at the molecular level, relative to wild type, microglia activation or microglial inflammation is not aggravated in the hippocampus of Kbk mice in their untreated state or in different treatments, including the TCF that has the greatest effect in influencing the progression and cellular state of Kbk microglia (FIG. 5A-B).

### Impact of TCF on Microglial Density, Filamentous Extensions, Soma Size and Overall Surveillance Phenotypes in the Hippocampus of Kbk Mice

Microglia are resident macrophages of myeloid origin in the brain, characterized by filaments/extensions that are connected to a cell body (or soma). Lengthening and branching of their filaments dominate at homeostasis to enable sensing and surveillance of brain areas. Activation of microglia in response to infection or injury results in shortening of extensions and enlargement of soma, to promote phagocytic functions and reduce surveillance. Ameboid morphologies are associated with neuroinflammation and ageing.

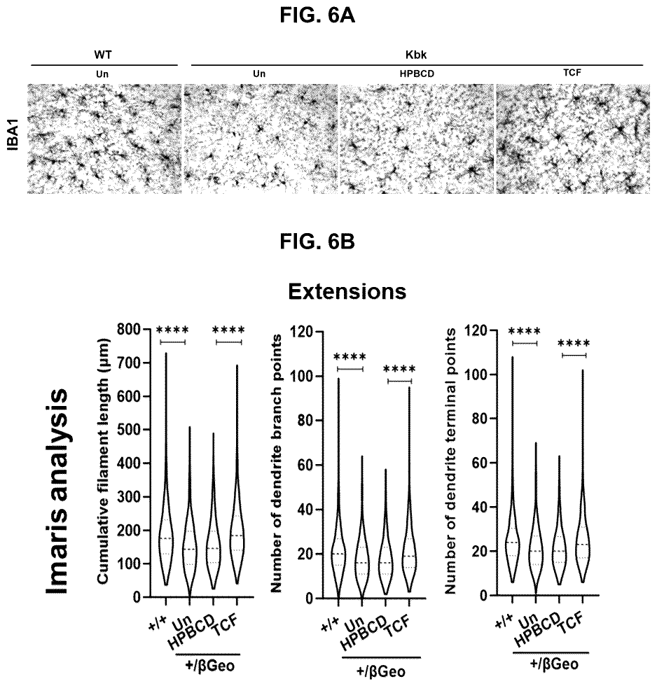

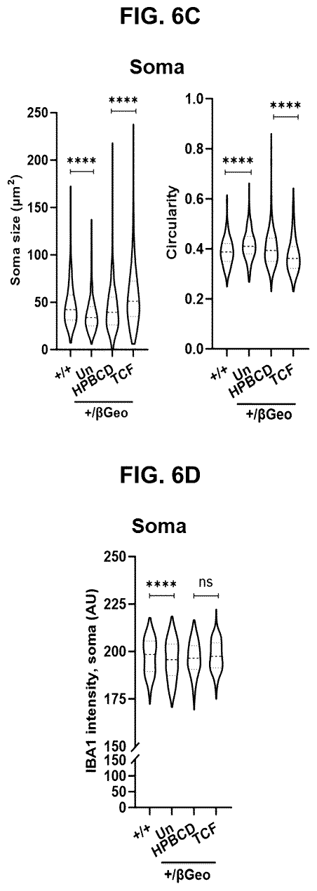

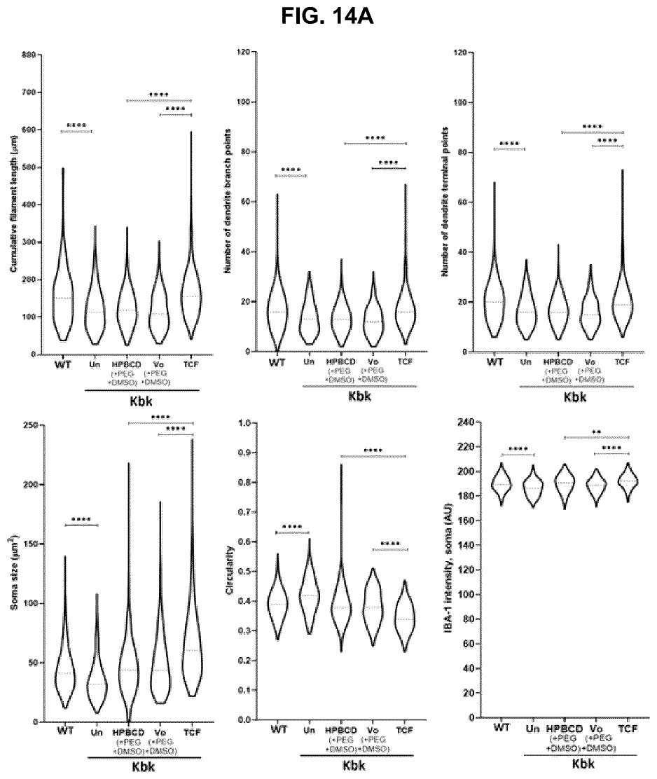

Since morphometric characterization is facilitated by automation, Imaris software was utilized to undertake quantitative, high-throughput analyses of 4-500 IBA1-stained hippocampal microglia from 3-month-old WT, Kbk animals and Kbk animals treated with TCF and appropriate control treatments. HP-β-CD (in DMSO+PEG), Vo (in DMSO+PEG). Compared to wild type (FIG. 6A) in Kbk mice (FIG. 6B), the maximal filament extension length, numbers of dendrite branch points and terminal points were reduced by 30-36%, while median levels of these parameters showed reduction of 17-20%. Maximal and median soma sizes in Kbk were also decreased (by 20%) while circularity showed a smaller increase by 6-8% (FIG. 6C). TCF promoted length and numbers of filament branch and terminal points by 42-64%. It increased the maximal and median size by 10-30% and reduced maximum and median roundness by 25 to 8%, respectively. In contrast, HP-β-CD (in PEG+DMSO) or Vo (in PEG+DMSO) had minimal or no significant effect (FIG. 14A-B), suggesting that only TCF specifically induced major changes in filament parameters and influenced soma (FIG. 14). The differences in IBA1 levels in mutants compared to wild type was 1-2% across groups, suggesting neither mutation nor treatments led to activation (FIG. 6D, and FIG. 14A upper right panels and FIG. 14B lower right panels). These changes were seen in both genders (FIG. 14) but with slight differences. The effects on soma size and circularity were variable between genders. In males, HP-β-CD (in PEG+DMSO), Vo (in PEG+DMSO) and TCF increased soma size to the untreated WT level, but the magnitude of TCF effect was maximal. In females, however, only TCF caused an increase in soma size (FIG. 14A-B) and soma size was reverted to WT levels by TCF. The opposite trend was observed for circularity (FIG. 14A-B, lower middle panels).

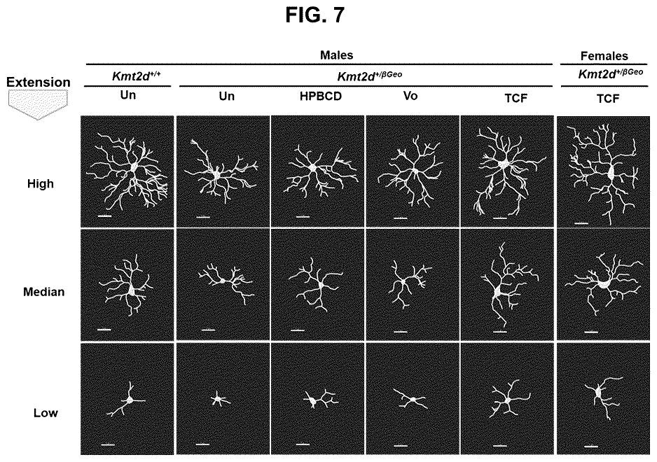

To better visualize the above changes, rendered Imaris models were used to display the range of extensions of hippocampal microglia from wild type mice and mutant mice, as well as the effects of different treatments (HP-β-CD (in DMSO+PEG), Vo in DMSO+PEG) and TCF) (FIG. 7). Wild type mice showed microglia with the highest level of ramified extensions branch and terminal points (High). Wild type mice also displayed median and low levels of filaments. The greatest filament length, branches, and end points of Kbk mice were comparable to the wild type ‘median’ group. Of the treatment groups, TCF-induced substantial increase in filament length, branch, and endpoints. These effects were seen in both genders and suggested that in conjunction with decrease in density, there may be reduced surveillance functions of hippocampal microglia in Kbk mice, and both can be restored by TCF (FIG. 6-7).

### Effects of Administration of TCF on Long Term Hippocampal Functions in Kbk Mice

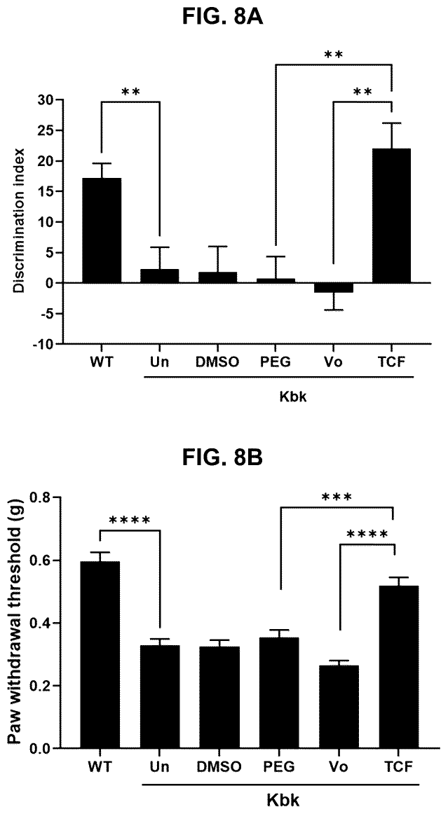

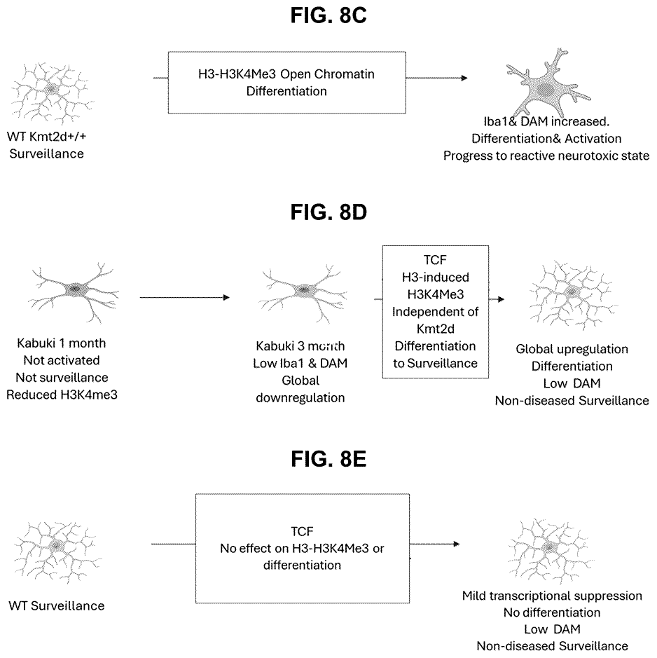

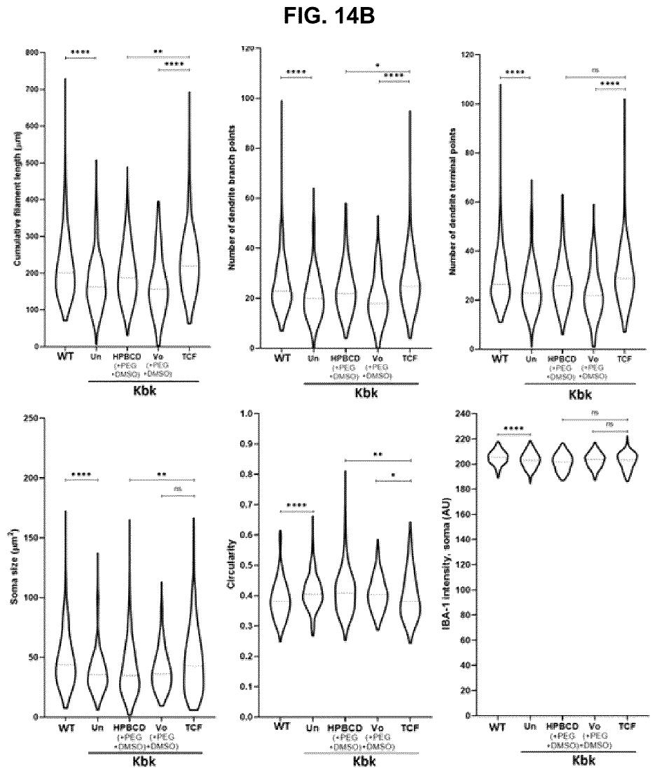

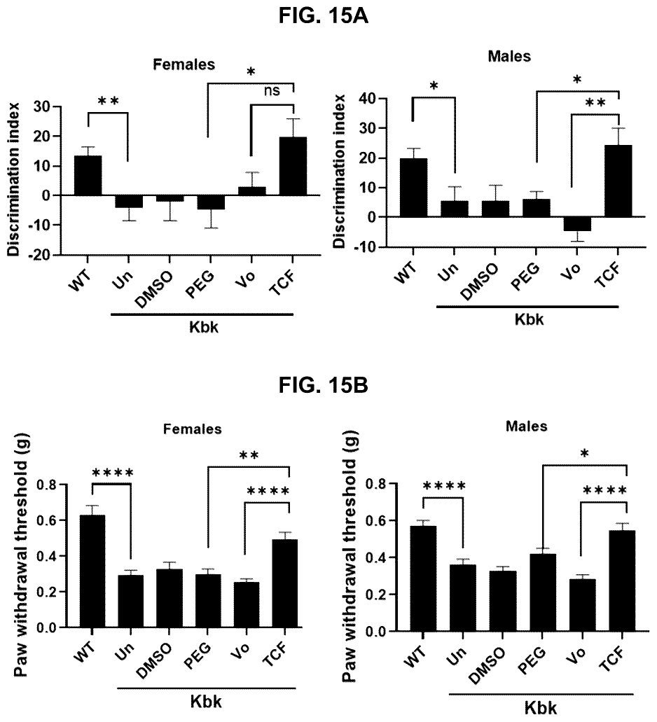

The Novel Object Recognition (NOR) test is a well-established and widely used measure of learning and memory in mice (and in prior work has been used to assess Kbk mice). Kbk animals showed reliable memory and learning defects as measured by a significant reduction in the Discrimination Index (DI) compared to wild type mice in the NOR test (FIG. 8A). DMSO, DMSO+PEG and Vo (in DMSO+PEG) had no significant effect. TCF significantly increased the DI to levels seen in wild type mice (FIG. 8A). Increased DI was also seen in both genders of TCF-treated mice (FIG. 15A).

For a second behavioral assay, the study explored tactile sensitivity/nociception since work suggests this is also a hippocampal function and tactile avoidance is reported in KS patients. The study therefore assessed mice for tactile responses using von Frey filaments. As shown (FIG. 8B), wild type mice displayed 50% paw withdrawal threshold of 0.61 g, which was reduced to 0.33 g in Kbk. DMSO, PEG (+DMSO) had no effect compared to controls. Vo (in PEG+DMSO) also had no effect, but TCF treatment improved the paw withdrawal threshold to 0.5 g. These effects were seen in both genders (FIG. 15A-B) establishing tactile hypersensitivity as a measurable symptomatic domain in KS mice. Moreover, TCF (but not its components) improved tactile responses to within 80% of that shown by wild type mice. It was not due to increased levels of Kmt2d transcript or the H3K4me3 mark, suggesting independence of Kmt2D.

The studies disclosed herein provide the first evidence for defects and disease in microglia in Kabuki Syndrome in mice. Microglia are known to become diseased in Alzheimer's, and other poly- and monogenetic neurological disorders as well as in cerebral infections. These microglia are highly activated, display disease associated microglia markers (DAMs) cause inflammatory pathology and become neurotoxic in the brain. Histone H3-dependent stimulation of the open chromatin marker H3K4m3 is well known to promote activation of microglia and their progression to diseased, neurotoxic states (see model in FIG. 8C). Surprisingly, diseased Kbk microglia are repressed in basic cellular functions of protein production and respiration, do not present capacity for inflammation, and even at three months post-natal, fail to display DAMs (summarized in FIG. 8D). TCF treatment drives diseased Kbk microglial trajectories towards differentiated states enriched for the H3K4me3 mark, suggesting that the TCF acts by driving open chromatin states to propagate differentiation of small-arrested Kbk microglia into surveillance forms, but in absence of increasing DAMs (FIG. 8D). Hence, it is proposed that prevention of DAM increase after elevating H3K4me3, as shown in FIG. 8D, may be a pathway of generating non-diseased surveillance forms in the brain.

These studies also underscore the specificity and therapeutic relevance of histone H3 modifications in addressing chromatin dysregulation. Although Vo can inhibit multiple HDACs, the finding that TCF selectively increases levels of histone H3, suggests that in microglia, it may preferentially inhibit HDAC3. Strong stimulation of the H3K4me3 mark upon HDAC3 inhibition has been shown to be associated with microglial differentiation and proliferation, but as indicated earlier in most neurodegenerative diseases this is associated with inflammation (FIG. 8C). In these studies in Kbk mice weekly administration of the TCF enables short exposure (2-4 h) of HDACi (2-4 h) with intermittent seven-day rest periods which may be important for selective action through histone H3. Although acetylation is not necessarily expected to increase levels of the target histone per se, small increases in the length of the S phase during microglial differentiation and proliferation may increase levels of histone H3. This may be amplified after repeated exposure to TCF and may further contribute to TCF-induced increase in H3K4me3. But TCF's induction of a large transcriptional response with positive amplitude in Kbk but not WT microglia suggests that open chromatin, may shield against aspects of inhibition of HDAC3 (and massive changes in gene transcription) as seen in healthy animals (FIG. 8E). This suggests that as mutant microglia become restored with H3K4me3 mark they may also become resistant to further remodeling by HDACi (particularly as drug concentrations drop precipitously after 2 h), limiting their advancement to activated states (as seen with genetic restoration of the chromatin mark).

No overt differences were observed in astrocytes in Kbk relative to wild type mice. Astrocytes are known to be triggered by activated microglia. The effects of TCF-induced development non-diseased surveillance microglia on astrocytes needs investigation, but since TCF-microglia present non-inflammatory signatures, they are not likely to trigger activation of neurotoxic astrocytes. Although TCF is sufficient to elicit robust transcriptional responses in Kbk microglia, it failed to stimulate post-natal neurogenesis. Prior studies showing stimulation of post-natal hippocampal neurogenesis by the HDACi AR-42, in Kbk mice, may be a consequence of sustained exposure after daily administration (at 10 mg/kg; that had to be discontinued after two weeks due to toxicity). In contrast, TCF (resulting in a single shot of 2-4 h of low micromolar concentration of Vo in the brain on a weekly basis) is well tolerated for many months in both mice and rats. The improved cognitive and tactile responses in KS mice, without boosting post-natal neurogenesis, suggest a role for microglia in hippocampal brain functions in Kbk mice and as targets for new therapies for KS as well as other chromatin disorders linked to intellectual disability.

What is claimed:

1. A method of treating tactile allodynia in a subject having Kabuki Syndrome, the method comprising administering to the subject a therapeutically effective amount of a composition comprising a histone deacetylase inhibitor, a polyethylene glycol (PEG), a cyclodextrin, and dimethyl sulfoxide (DMSO), thereby treating the tactile allodynia in the subject.

2. The method of claim 1, wherein the histone deacetylase inhibitor comprises vorinostat, belinostat, romidepsin, dacinostat, panobinostat, givinostat, pyroxamide, trichostatin A, m-carboxycinnamic acid bis-hydroxamide (CBHA), or combinations thereof.

3. The method of claim 1, wherein the histone deacetylase inhibitor comprises vorinostat.

4. The method of claim 1, wherein the PEG has an average molecular weight of about 200 Da to about 600 Da.

5. The method of claim 1, wherein the PEG comprises PEG-400.

6. The method of claim 1, wherein the cyclodextrin has an average molecular weight of about 900 Da to about 1500 Da.

7. The method of claim 1, wherein the cyclodextrin comprises 2-hydroxypropyl-β-cyclodextrin (HP-β-CD), dimethyl-β-cyclodextrin, hydroxypropyl-α-cyclodextrin, hydroxypropyl-γ-cyclodextrin, or combinations thereof.

8. The method of claim 1, wherein the cyclodextrin comprises 2-hydroxypropyl-β-cyclodextrin (HP-β-CD).

9. The method of claim 1, wherein the composition comprises vorinostat, PEG-400, 2-hydroxypropyl-β-cyclodextrin (HP-β-CD), and DMSO.

10. The method of claim 1, wherein the composition comprises about 10 wt % to about 40 wt % histone deacetylase inhibitor; about 15 wt % to about 50 wt % PEG; about 20 wt % to about 50 wt % cyclodextrin; and about 5 wt % to about 10 wt % DMSO.

11. The method of claim 1, wherein the subject is administered a dose of the composition of about 10 mg/kg to about 1000 mg/kg.

12. The method of claim 1, wherein the composition is administered to the subject over a period of about 1 week to about 12 months.

13. The method of claim 1, wherein the composition is administered to the subject once daily, twice daily, once every other day, once weekly, twice weekly, once biweekly, once monthly, twice monthly, once every two or three months, or combinations thereof.

14. The method of claim 1, wherein administering the composition to the subject comprises intramuscular administration, subcutaneous administration, intravenous administration, intrathecal administration, intraperitoneal administration, or combinations thereof.

15. The method of claim 1, further comprising performing one or more mechanical allodynia tests, heightened sensitivity to touch tests, mechanical pain sensitivity tests, or quantitative sensory tests to assess the tactile allodynia in the subject.

16. The method of claim 15, wherein the one or more mechanical allodynia tests, heightened sensitivity to touch tests, mechanical pain sensitivity tests, or quantitative sensory tests comprise von Frey filament testing, brush-evoked dynamic mechanical allodynia testing, cotton swab or light touch stimulation testing, pinprick or punctate mechanical stimulation testing, mechanical withdrawal threshold testing, mechanical pain threshold testing, quantitative sensory testing assessing mechanical detection threshold, mechanical pain threshold, mechanical pain sensitivity, or dynamic mechanical allodynia, or combinations thereof.

17. The method of claim 1, wherein the method increases proliferation of microglia cells in the subject relative to before the subject is administered the composition.

18. The method of claim 1, wherein the method increases one or more of histone H3 acetylation (H3ac) or histone H3 lysine 4 trimethylation (H3K4me3) in brain cells of the subject relative to before the subject is administered the composition.

19. The method of claim 1, wherein the method improves one or more of cognition, memory, sensory responses, or learning in the subject relative to before the subject is administered the composition.

20. The method of claim 1, wherein the method does not affect post-natal hippocampal neurogenesis in the subject.

21. A method of improving one or more of cognition, memory, sensory responses, or learning in a subject having Kabuki Syndrome, the method comprising administering to the subject a therapeutically effective amount of a composition comprising a histone deacetylase inhibitor, a polyethylene glycol (PEG), a cyclodextrin, and dimethyl sulfoxide (DMSO), thereby improving one or more of cognition, memory, sensory responses, or learning in the subject.

22. The method of claim 21, wherein the composition comprises vorinostat, PEG-400, 2-hydroxypropyl-β-cyclodextrin (HP-β-CD), and DMSO.

23. The method of claim 21, further comprising performing one or more standardized neuropsychological tests to assess one or more of cognition, memory, sensory responses, or learning in the subject.

24. A kit for treating tactile allodynia in a subject having Kabuki Syndrome, the kit comprising:
a composition comprising a histone deacetylase inhibitor, a polyethylene glycol (PEG), a cyclodextrin, and dimethyl sulfoxide (DMSO);
optionally, administration materials for delivery to a subject;
optionally, buffers and receptacles; and
optionally, one or more of packaging, a label, or instructions for use.
